(19) United States
(12) Patent Application Publication
(10) Pub. No.: US 2025/0297296 A1
(43) Pub. Date: Sep. 25, 2025

(54) NEW SIALYLTRANSFERASES FOR IN VIVO SYNTHESIS OF LST-A

(71) Applicant: DSM IP Assets B.V., HEERLEN (DK)

(72) Inventor: Manos PAPADAKIS, Hørsholm (DK)

(21) Appl. No.: 18/843,530

(22) PCT Filed: Mar. 1, 2023
(86) PCT No.: PCT/EP2023/055146

§ 371 Date: Sep. 3, 2024

(30) Foreign Application Priority Data
Mar. 2, 2022 (DK) PA202270078

(51) Int. Cl. C12P 19/18; C12N 9/10; C12P 19/02

(52) U.S. Cl. CPC C12P 19/18; C12N 9/1048; C12N 9/1051; C12P 19/02; C12Y 204/01222

(57) ABSTRACT

The present disclosure relates to the production of sialylated Human Milk Oligosaccharides (HMOs), in particular to the production of sialyl-lacto-N-tetraose a (LST-a), from precursor oligosaccharides and the genetic engineering of suitable cells for use in said production, as well as to methods for producing said sialylated HMOs.

### CROSS-REFERENCE TO RELATED APPLICATIONS

This application is a national stage entry pursuant to 35 U.S.C. § 371 of International Application No. PCT/EP2023/055146, filed on Mar. 1, 2023, which claims priority to Denmark Application No. PA202270078, filed on Mar. 2, 2022, the entire contents of all of which are hereby incorporated by reference in their entirety.

### SEQUENCE LISTING

The computer-readable Sequence Listing submitted on Apr. 14, 2025 and identified as follows: 85,152 bytes ST.26 XML document file named “032991-8020 Sequence Listing.xml,” created Apr. 14, 2025, is incorporated herein by reference in its entirety.

### FIELD

The present disclosure relates to the production of sialylated Human Milk Oligosaccharides (HMOs), in particular to the production of sialyl-lacto-N-tetraose a (LST-a), and to genetically engineered cells suitable for use in said production.

### BACKGROUND

The design and construction of bacterial cell factories to produce sialylated Human Milk Oligosaccharides (HMOs), especially for more complex sialylated Human Milk Oligosaccharides (HMOs), is of paramount importance to provide innovative and scalable solutions for the more complex products of tomorrow.

To this end, rational strain engineering principles are commonly applied to single bacterial cells. Such principles usually refer to a) the introduction of a desired biosynthetic pathway to the host, b) the increase of the cellular pools of relevant activated sugars required as donors in the desired reactions, c) the enhancement of lactose import by the native lactose permease LacY and d) the introduction of suitable glycosyltransferases to facilitate the biosynthetic production of sialylated oligosaccharides (for review see Bych et al 2019, Current Opinion in Biotechnology 56:130-137).

Production of sialylated HMOs has e.g., been disclosed in WO2007/101862, describing the modifications needed to produce e.g., 3′-SL from a non-pathogenic microorganism without having to supply sialic acid to the culture resulting in a cheaper large-scale production of sialylated HMOs.

WO2019/020707 and WO2019/228993 in turn describe examples of sialyltransferases expressed in a genetically modified cell, which are capable of producing sialylated HMOs. The sialyltransferases disclosed therein, however, only produce no or minor amounts of complex sialylated HMOs, and show high byproduct formation.

Production of sialylated HMOs, can be hampered by side-activities of the sialyltransferases in the production strain, which may affect the ability of the cell to grow robustly even in the absence of substrate which is in turn reflected in poor yields of the sialylated HMO product.

In summary, production of sialylated HMOs, especially more complex sialylated Human Milk Oligosaccharides (HMOs), is often hampered by low production yield of the desired sialylated HMO as compared to other HMO products present after fermentation, such as HMO precursor products, as well as the simultaneous formation of other sialylated HMO species (HMO by-products), which in turn requires laborious separation procedures. Thus, sialyltransferases that are more specific towards one or more specific sialylated HMOs, in particular towards one or more specific complex sialylated HMO, are needed to lower byproduct formation and to simplify product purification.

### SUMMARY

The present disclosure relates to a genetically modified cell comprising a recombinant nucleic acid sequence encoding an enzyme with α-2,3-sialyltransferase activity, capable of transferring sialic acid from an activated sugar to the terminal galactose of LNT (acceptor) and/or to the galactose of lactose (acceptor). The genetically modified cell is capable of producing HMO, wherein at least 9% of the total molar HMO content produced by the cell is LST-a.

In particular, the present disclosure relates to a genetically modified cell comprising a recombinant nucleic acid sequence encoding an enzyme selected from the group consisting of Ccol2, Cjej1, Csub1, Chepa and Clari1 with an amino acid sequence with at least 80% identity to an amino acid sequence selected from the group consisting of SEQ ID NO: 1, 2, 3, 4 and 5, respectively, and wherein said cell produces at least one sialylated Human Milk Oligosaccharide (HMO). The sialylated HMO is typically LST-a and/or 3′SL, such that at least 9% of the total molar HMO content produced by the cell is LST-a. Typically, the level of 3′SL produced is below 20%, such as below 10% of the total molar content of the HMOs produced by said cell.

The genetically modified cell according to the present disclosure can further comprise a promoter element that controls the expression of the recombinant nucleic acid encoding an enzyme with α-2,3-sialyltransferase activity. The sialyltransferase may e.g., be under the control of a promoter selected from the group consisting of PglpF, Plac, PmglB_70UTR PglpA_70UTR and PglpT_70UTR and variants thereof with a nucleic acid sequence selected from the group consisting of SEQ ID NOs 15-23 and 41 to 55, respectively. Preferably, the recombinant nucleic acid encoding an enzyme with α-2,3-sialyltransferase is under control of a strong promoter selected from the group consisting of SEQ ID NOs 15, 20, 21, 22, 23, 41, 42, 43, 44, 45, 46, 47, 48, 49, 50, 51, and 52.

The genetically modified cell according to the present disclosure can further comprise a nucleic acid sequence encoding an MFS transporter protein capable of exporting the sialylated HMO into the extracellular medium.

The genetically modified cell according to the present disclosure can further comprise at least one recombinant nucleic acid sequence encoding at least one glycosyltransferase capable of transferring a glycosyl residue from a glycosyl donor to an acceptor oligosaccharide to produce a precursor of the sialylated human milk oligosaccharide product, such as LNT, or to further decorate a sialylated human milk oligosaccharide to produce a more complex sialylated human milk oligosaccharide.

Further, the genetically modified cell according to the present disclosure typically comprises a recombinant nucleic acid sequence encoding a β-1,3-N-acetyl-glucosaminyl-transferase, such as LgtA from Neisseria meningitidis and/or a recombinant nucleic acid sequence encoding a β-1,3-galactosyltransferase, such as GaITK from Helicobacter pylori.

The genetically modified cell according to the present disclosure can comprise a biosynthetic pathway for making a sialic acid sugar nucleotide, such as CMP-Neu5Ac. Said sialic acid sugar nucleotide pathway can be encoded by the nucleic acid sequence encoding neuBCA from Campylobacter jejuni (SEQ ID NO: 38). The nucleic acid sequence encoding neuBCA, can be encoded from a high-copy plasmid bearing the neuBCA operon.

The genetically modified cell according to the present disclosure can be a microorganism, such as a bacterium or a fungus, wherein said fungus can be selected from a yeast cell, such as of the genera Komagataella, Kluyveromyces, Yarrowia, Pichia, Saccaromyces, Schizosaccharomyces or Hansenula, or from a filamentous fungous of the genera Aspargillus, Fusarium or Thricoderma, and said bacterium can be selected from the exemplified group consisting of Escherichia sp., Bacillus sp., lactobacillus sp. and Campylobacter sp. Accordingly, the genetically modified cell according to the present disclosure can be E coli.

The genetically modified cell of the present disclosure can be used in the production of a sialylated HMO.

Accordingly, the present disclosure also relates to a method for producing a sialylated human milk oligosaccharide (HMO), said method comprising culturing a genetically modified cell according to the present disclosure.

In addition, the disclosure also relates to a nucleic acid construct encoding an enzyme with α-2,3-sialyltransferase activity, such as an enzyme selected from the group consisting of Ccol2, Cjej1, Csub1, Chepa and Clari1, wherein the enzyme encoding sequence is preferably under the control of a promoter sequence, such as a promoter selected from the group consisting of PglpF, Plac, PmglB_70UTR, PglpA_70UTR and PglpT_70UTR and variants thereof (SEQ ID NOs 15-23 and SEQ ID NO: 41-55). Said nucleic acid construct is typically used in a host cell for producing a sialylated HMO, such as LST-a and/or 3′SL.

Various exemplary embodiments and details are described hereinafter, with reference to the figures and sequences when relevant. It should be noted that the figures are only intended to facilitate the description of the embodiments. They are not intended as an exhaustive description of the disclosure or as a limitation on the scope of the disclosure. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

### BRIEF DESCRIPTION OF THE FIGURES

FIG. 1

Cells expressing an enzyme with an α-2,3-sialyltransferase activity that produce a molar content of LST-a (in percentage, % of total HMO) that exceeds LST-a levels produced by cells expressing CstI, CstII and PM70, which are known in the prior art to be able to sialylate LNT.

### DETAILED DESCRIPTION

The present disclosure approaches the biotechnological challenges of in vivo HMO production, in particular of sialylated HMOs that contain at least one sialyl monosaccharide, such as the sialylated HMOs 3′SL and LST-a. The present disclosure offers specific strain engineering solutions to produce specific complex sialylated HMOs, in particular LST-a, by exploiting the substrate specificity towards the terminal galactose moiety on LNT and activity of the α-2,3-sialyltransferases of the present disclosure.

In other words, a genetically modified cell covered by the present disclosure expresses genes encoding key enzymes for sialylated HMO biosynthesis, in some embodiments along with one or more genes encoding a biosynthetic pathway for making a sialic acid sugar nucleotide, such as the neuBCA operon from Campylobacter jejuni shown in SEQ ID NO: 38, which enables the cell to produce a sialylated oligosaccharide from one or more oligosaccharide substrates, such as lactose, LNT-II and/or LNT, and one or more nucleotide-activated sugars, such as glucose-UDP-GlcNac, GDP-fucose, UDP-galactose, UDP-glucose, UDP-N-acetylglucosamine, UDP-N-acetylgalactosamine and CMP-N-acetylneuraminic acid.

In particular, the sialylated HMO(s) produced is LST-a and/or 3′SL.

The advantage of using any one of the α-2,3-sialyltransferases of the present disclosure in the present context is their ability to recognize and sialylate, not only lactose to generate 3′SL, but also larger oligosaccharides, such as LNT, to generate LST-a. The enzymes presented here not only provide high LST-a titers, but they are also more specific for the LNT acceptor rather than the lactose acceptor. In particular, the present disclosure describes α-2,3-sialyltransferases that are more active on the terminal galactose of LNT than α-2,3-sialyltransferases described in the prior art, such as CstI, CstII and PM70 (see WO2019/020707). The traits of the α-2,3-sialyltransferases described herein are therefore well-suited for high-level industrial production of LST-a and the simultaneous minimal or lesser formation of other sialylated HMOs, such as 3′SL and other by-product HMOs.

The genetically modified cells of the present disclosure, which express a more selective α-2,3-sialyltransferase with high LNT specificity, for the first time enable the production of high titers of LST-a, at the same time reducing the titers of undesired other sialylated HMOs, such as 3′SL to at the most 20%, such as no more than 10% of the total molar content of the HMOs produced by said cells, and other impurities. Thereby, the present disclosure enables a more efficient LST-a production, which is highly beneficial in biotechnological production of more complex sialylated HMOs, such as LST-a.

In the following sections, individual elements of the disclosure and in particular of the genetically modified cell is described, it is understood that these elements can be combined across the individual sections.

### Oligosaccharides

In the present context, the term “oligosaccharide” means a sugar polymer containing at least three monosaccharide units, i.e., a tri-, tetra-, penta-, hexa- or higher oligosaccharide. The oligosaccharide can have a linear or branched structure containing monosaccharide units that are linked to each other by interglycosidic linkages. Particularly, the oligosaccharide comprises a lactose residue at the reducing end and one or more naturally occurring monosaccharides of 5-9 carbon atoms selected from aldoses (e.g., glucose, galactose, ribose, arabinose, xylose, etc.), ketoses (e.g., fructose, sorbose, tagatose, etc.), deoxysugars (e.g. rhamnose, fucose, etc.), deoxy-aminosugars (e.g. N-acetyl-glucosamine, N-acetyl-mannosamine, N-acetyl-galactosamine, etc.), uronic acids and ketoaldonic acids (e.g. N-acetylneuraminic acid). Preferably, the oligosaccharide is an HMO.

### Human Milk Oligosaccharide (HMO)

Preferred oligosaccharides of the disclosure are human milk oligosaccharides (HMOs).

The term “human milk oligosaccharide” or “HMO” in the present context means a complex carbohydrate found in human breast milk. The HMOs have a core structure comprising a lactose unit at the reducing end that can be elongated by one or more beta-N-acetyl-lactosaminyl and/or one or more beta-lacto-N-biosyl unit, and this core structure can be substituted by an alpha-L-fucopyranosyl and/or an alpha-N-acetyl-neuraminyl (sialyl) moiety. HMO structures are e.g., disclosed by Xi Chen in Chapter 4 of Advances in Carbohydrate Chemistry and Biochemistry 2015 vol 72.

The present disclosure focuses on sialylated HMO's, which are generally acidic. Examples of acidic HMOs include 3′-sialyllactose (3′SL), 6′-sialyllactose (6′SL), 3-fucosyl-3′-sialyllactose (FSL), 3′-O-sialyllacto-N-tetraose a (LST-a), fucosyl-LST-a (FLST-a), 6′-O-sialyllacto-N-tetraose b (LST-b), fucosyl-LST b (FLST b), 6′-O-sialyllacto-N-neotetraose (LST-c), fucosyl-LST-c (FLST-c), 3′-O-sialyllacto-N-neotetraose (LST-d), fucosyl-LST d (FLST-d), sialyl-lacto-N-hexaose (SLNH), sialyl-lacto-N-neohexaose I (SLNH-I), sialyl-lacto-N-neohexaose II (SLNH-II) and disialyl-lacto-N-tetraose (DSLNT).

In the context of the present disclosure, complex HMOs are composed of at least 4 monosaccharide units, preferably at least 5 monosaccharide units. Preferably, in one embodiment, a complex HMO is one that require at least two different glycosyltransferase activities to be produced from lactose as the initial substrate, e.g., the formation of LST-a requires an alpha-2,3-sialyltransferase, a β-1,3-N-acetyl-glucosaminyl-transferase and a β-1,3-galactosyltransferase.

In one aspect according to the present disclosure, the human milk oligosaccharide (HMO) is an acidic HMO such as a sialylated HMO. The sialylated HMO in one aspect comprises at least three monosaccharide units, such as three, four or five monosaccharide units.

In one aspect of the present disclosure, the sialylated human milk oligosaccharide (HMO) produced by the cell is a sialylated HMO selected from the list consisting of 3′SL, DSLNT, and LST-a. In a further aspect of the present disclosure, the sialylated human milk oligosaccharide (HMO) produced by the cell is an HMO of at least five monosaccharide units, such as LST-a.

Production of these HMO's may require the presence of two or more glycosyltransferase activities, in particular if starting from lactose as the acceptor oligosaccharide.

### An Acceptor Oligosaccharide

A genetically modified cell according to the present disclosure comprises a recombinant nucleic acid sequence encoding an enzyme with α-2,3-sialyltransferase activity capable of transferring sialic acid from an activated sugar to the terminal galactose of an acceptor oligosaccharide.

In the context of the present disclosure, an acceptor oligosaccharide is an oligosaccharide that can act as a substrate for a glycosyltransferase capable of transferring a glycosyl moiety from a glycosyl donor to the acceptor oligosaccharide. The glycosyl donor is preferably a nucleotide-activated sugar as described in the section on “glycosyltransferases”. Preferably, the acceptor oligosaccharide is a precursor for making a more complex HMO and can also be termed the precursor molecule.

The acceptor oligosaccharide can be either an intermediate product of the present fermentation process, an end-product of a separate fermentation process employing a separate genetically modified cell, or an enzymatically or chemically produced molecule.

In the present context, said acceptor oligosaccharide for the α-2,3-sialyltransferase is preferably lacto-N-neotetraose (LNT), which is produced from the precursor molecules lactose (e.g., acceptor for the β-1,3-N-acetyl-glucosaminyl-transferase) and/or lacto-N-triose (LNT-II) (e.g., acceptor for the β-1,3-galactosyltransferase). The precursor molecule is preferably fed to the genetically modified cell which is capable of producing LNT from the precursor.

### Glycosyltransferases

The genetically modified cell according to the present disclosure comprises at least one recombinant nucleic acid sequence encoding at least one glycosyltransferase capable of transferring a sialyl residue from a sialyl donor to an acceptor oligosaccharide to synthesize a sialylated human milk oligosaccharide product, i.e., a sialyltransferase.

The genetically modified cell according to the present disclosure may comprise at least one further recombinant nucleic acid sequence encoding at least one glycosyltransferase capable of transferring a glycosyl residue from a glycosyl donor to an acceptor oligosaccharide. Preferably, the additional glycosyltransferase(s) enables the genetically modified cell to synthesize LNT from a precursor molecule, such as lactose or LNT-II. The additional glycosyltransferase may also be capable of further decorating e.g., LST-a to generate DSLNT, or a 3′SL molecule to generate DSL.

The additional glycosyltransferase is preferably selected from the group consisting of, galactosyltransferases, gIucosaminyltransferases, sialyltransferases, N-acetylglucosaminyl transferases and N-acetylglucosaminyl transferases.

In one aspect, the sialyltransferase in the genetically modified cell of the present disclosure is an α-2,3-sialyltransferase. Preferably, the α-2,3-sialyltransferase is capable of transferring a sialic acid unit onto the terminal galactose of an LNT molecule. It is even more preferred that the α-2,3-sialyltransferase of the present disclosure has a higher affinity for the terminal galactose moiety in LNT as compared to the terminal galactose moiety in lactose.

In one embodiment, the α-2,3-sialyltransferase of the present disclosure results in an LST-a formation that exceeds the formation of 3′SL when using lactose as the starting substrate, preferably the molar % of LST-a is at least 1.5 times above the molar % of 3′SL, more preferred the molar % of LST-a is 2 times above the molar % of 3′SL, even more preferred, the molar % of LST-a is 3 times above the molar % of 3′SL.

In the present disclosure, the at least one functional enzyme (α-2,3-sialyltransferase) capable of transferring a sialyl moiety from a sialyl donor to an acceptor oligosaccharide can be selected from the list consisting of Ccol2, Cjej1, Csub1, Chepa and Clari1 (table 1). These enzymes can e.g., be used to produce 3′SL and/or LST-a, respectively.

In one embodiment, the α-2,3-sialyltransferase described herein is further combined with a β-1,3-galactosyltransferase, such as galTK from Helicobacter pylori. In a further embodiment, a third enzyme is added, such as a β-1,3-N-acetyl-glucosaminyl-transferase, e.g., LgtA from Neisseria meningitidis.

In one embodiment, the α-2,3-sialyltransferase described herein is further combined with a β-1,3-galactosyltransferase, such as galTK from Helicobacter pylori and a β-1,3-N-acetyl-glucosaminyl-transferase, e.g., LgtA from Neisseria meningitidis. In this embodiment the cell is able to produce LST-a from lactose as the initial substrate.

Exemplified glycosyltransferases are preferably selected from the glycosyltransferases described below.

### α-2,3-sialyltransferase

An alpha-2,3-sialyltransferase refers to a glycosyltransferase that catalyzes the transfer of sialyl from a donor substrate, such as CMP-N-acetylneuraminic acid, to an acceptor molecule in an alpha-2,3-linkage. Preferably, an alpha-2,3-sialyltransferase used herein does not originate in the species of the genetically engineered cell, i.e., the gene encoding the alpha-2,3-sialyltransferase is of heterologous origin and is selected from an alpha-2,3-sialyltransferase identified in table 1. In the context of the present disclosure, the acceptor molecule for the alpha-2,3-sialyltransferase is lactose and/or an acceptor oligosaccharide of at least four monosaccharide units, e.g., LNT. Heterologous alpha 2,3-sialyltransferases that are capable of transferring a sialyl moiety onto LNT are known in the art, three of which are identified in table 1.

The α-2,3-sialyltransferases investigated in the present application are listed in table 1. The sialyltransferase can be selected from an amino acid sequence with at least 80%, such as at least 85%, such as at least 90%, such as at least 95%, or such as at least 99% identity to the amino acid sequence of any one of the alpha-2,3-sialyltransferases listed in table 1.

TABLE 1
List of alpha-2,3-sialyltransferase enzymes capable of producing LST-a

| Enzyme | | SEQ | | |
|---|---|---|---|---|
| Name | GenBank ID | ID NO: | Origin | Ref |
| Ccol2 | EAH6554614.1 | 1 | Campylobacter coli | |
| Cjej1 | EBD1936710.1 | 2 | Campylobacter jejuni | |
| Csub1 | WP_039664428.1 | 3 | Campylobacter | |
| | | | subantarcticus | |
| Chepa | WP_066776435.1 | 4 | Campylobacter hepaticus | |
| Clari1 | EGK8106227.1 | 5 | Campylobacter lari | |
| Ccol | WP_075498955.1 | 6 | Campylobacter coli | |
| MhnNBse | WP_176810284.1 | 7 | Mannheimia (multispecies) | |
| Pmult | WP_005753497.1 | 8 | Pasteurella multocida | |
| Neigon | AAW89748.1 | 9 | Neisseria gonorrhoeae FA | |
| | | | 1090 | |
| Poral | WP_101774487.1 | 10 | Pasteurella oralis | |
| Cinf1 | WP_011272254.1 | 11 | Haemophilus influenzae | |
| PM70 | AAK03258.1 | 12 | Pasteurella multocida subsp. | WO2019/020707 |
| | | | Pm70 | |
| CstI | AAF13495.1 | 13 | Campylobacter jejuni | WO2019/020707 |
| CstII | AAF31771.1 | 14 | Campylobacter jejuni | WO2019/020707 |

The GenBank ID's reflect the full length enzymes, in the present disclosure truncated or mutated versions may have been used, these are represented by the sequences indicated by the SEQ ID NOs.

Example 1 of the present disclosure has identified the heterologous alpha-2,3-sialyltransferases Ccol2, Cjej1, Csub1, Chepa and Clari1 (SEQ ID NO: 1, 2, 3, 4 and 5, respectively), which are capable of producing higher LST-a titers when introduced into an LNT producing cell, than the known PM70, CstI and CstII.

In the examples Ccol2, Cjej1, Csub1, Chepa and Clari1 are used in combination with LgtA from Neisseria meningitidis and galTK from Helicobacter pylori to produce a mixture of LST-a and 3′SL starting from lactose as substrate. Ccol2, Cjej1, Csub1, Chepa or Clari1 may alternatively be combined with galTK from Helicobacter pylori to produce LST-a starting from LNT-II as substrate, this could eliminate the formation of 3′SL. Additionally, Ccol2, Cjej1, Csub1, Chepa or Clari1 may be sufficient to produce LST-a when starting from LNT.

If desired, the alpha-2,3-sialyltransferases identified in table 1, may also be used in a modified strain without β-1,3-N-acetyl-glucosaminyl-transferase and β-1,3-galactosyltransferase activity, resulting in the production of 3′SL without the presence of LST-a when using lactose as substrate.

In one embodiment, the enzyme with α-2,3-sialyltransferase activity is Ccol2 from Campylobacter coli comprising or consisting of the amino acid sequence of SEQ ID NO: 1 or an amino acid sequence with at least 80%, such as at least 85%, such as at least 90%, such as at least 95%, or such as at least 99% identity to SEQ ID NO: 1.

In another embodiment, the enzyme with α-2,3-sialyltransferase activity is Cjej1 from Campylobacter jejuni comprising or consisting of the amino acid sequence of SEQ ID NO: 2 or an amino acid sequence with at least 80%, such as at least 85%, such as at least 90%, such as at least 95%, or such as at least 99% identity to SEQ ID NO: 2.

In another embodiment, the enzyme with α-2,3-sialyltransferase activity is Csub1 from Campylobacter subantarcticus comprising or consisting of the amino acid sequence of SEQ ID NO: 3 or an amino acid sequence with at least 80%, such as at least 85%, such as at least 90%, such as at least 95%, or such as at least 99% identity to SEQ ID NO: 3.

In another embodiment, the enzyme with α-2,3-sialyltransferase activity is Chepa from Campylobacter hepaticus comprising or consisting of the amino acid sequence of SEQ ID NO: 4 or an amino acid sequence with at least 80%, such as at least 85%, such as at least 90%, such as at least 95%, or such as at least 99% identity to SEQ ID NO: 4, and/or In another embodiment, the enzyme with α-2,3-sialyltransferase activity is Clari1 from Campylobacter clari comprising or consisting of the amino acid sequence of SEQ ID NO: 5 or an amino acid sequence with at least 80%, such as at least 85%, such as at least 90%, such as at least 95%, or such as at least 99% identity to SEQ ID NO: 5.

### β-1,3-N-acetyl-glucosaminyl-transferase

A β-1,3-N-acetyl-glucosaminyl-transferase is any protein which comprises the ability of transferring the N-acetyl-glucosamine of UDP-N-acetyl-glucosamine to lactose or another acceptor molecule, in a beta-1,3-linkage. Preferably the β-1,3-N-acetyl-glucosaminyl-transferase used herein does not originate in the species of the genetically engineered cell, i.e., the gene encoding the β-1,3-galactosyltransferase is of heterologous origin. In the context of the present disclosure, the acceptor molecule is either lactose or an oligosaccharide of at least four monosaccharide units, e.g., LNT, or more complex HMO structures.

β-1,3-N-acetylglucosaminyltransferases can be obtained from a number of sources, e.g., the IgtA genes described from N. meningitidis strains (GenBank protein Accession ID's AAF42258.1, WP_002248149.1 or WP_033911473.1 or ELK60643.1) or from N. gonorrhoeae (GenBank protein Accession nr.'s ACF31229.1 or AAK70338.1) or from Haemophilus ducreyi (GenBank protein Accession AAN05638.1) or from Pasteurella multocida (GenBank protein Accession AAK02595.1) or from Neisseria cinerea (GenBank protein Accession EEZ72046.1).

In one embodiment, the recombinant nucleic acid sequence encoding a β-1,3-N-acetylglucosaminyltransferase comprises or consists of the amino acid sequence of SEQ ID NO: 39 (LgtA from N. meningitidis) or a functional homologue thereof with an amino acid sequence with at least 80%, such as at least 85%, such as at least 90%, such as at least 95%, or such as at least 99% identity to SEQ ID NO: 39.

For the production of LNT from lactose as substrate, the LNT-II precursor is formed using a β-1,3-N-acetylglucosaminyltransferase. In one embodiment the genetically modified cell comprises a β-1,3-N-acetylglucosaminyltransferase gene, or a functional homologue or fragment thereof, to produce the intermediate LNT-II from lactose.

Some of the examples below use the heterologous β-1,3-N-acetyl-glucosaminyl-transferase named LgtA from Neisseria meningitidis or a variant thereof.

### β-1,3-galactosyltransferase

A β-1,3-galactosyltransferase is any protein that comprises the ability of transferring the galactose of UDP-Galactose to a N-acetyl-glucosaminyl moiety to an acceptor molecule in a beta-1,3-linkage. Preferably, a β-1,3-galactosyltransferase used herein does not originate in the species of the genetically engineered cell i.e., the gene encoding the β-1,3-galactosyltransferase is of heterologous origin. In the context of the present disclosure the acceptor molecule, is an acceptor saccharide, e.g., LNT-II, or more complex HMO structures.

The examples below use the heterologous β-1,3-galactosyltransferase named GaITK or a variant thereof, to produce e.g., LST-a in combination with other glycosyl transferases.

β-1,3-galactosyltransferases can be obtained from any one of a number of sources, e.g., the gaITK gene from H. pylori as described, (homologous to GenBank protein Accession BD182026.1) or the WbgO gene from E. coli 055:H7 (GenBank Accession WP_000582563.1) or the jhp0563 gene from H. pylori (GenBank Accession AEZ55696.1).

In one embodiment, the recombinant nucleic acid sequence encoding a β-1,3-galactosyltransferases comprises or consists of the amino acid sequence of SEQ ID NO: 40 (galTK from H. pylori) or a functional homologue thereof with an amino acid sequence with at least 80%, such as at least 85%, such as at least 90%, such as at least 95%, or such as at least 99% identity to SEQ ID NO: 40.

To produce LNT form an LNT-1l precursor, a β-1,3-galactosyltransferase is needed. In one embodiment, the genetically modified cell comprises a β-1,3-galactosyltransferase gene, or a functional homologue or fragment thereof.

Below are examples of genetically modified strains according to the present disclosure with specific combinations of glycosyl transferases that will lead to production of LST-a using lactose as initial substrate.

In one example, LgtA from Neisseria meningitidis is used in combination with galTK from Helicobacter pylori and Ccol2 from Campylobacter coli to produce LST-a starting from lactose as initial substrate.

In one example, LgtA from Neisseria meningitidis is used in combination with galTK from Helicobacter pylori and Cjej1 from Campylobacter jejuni to produce LST-a starting from lactose as initial substrate.

In one example, LgtA from Neisseria meningitidis is used in combination with galTK from Helicobacter pylori and Csub1 from Campylobacter subantarcticus to produce LST-a starting from lactose as initial substrate.

In one example, LgtA from Neisseria meningitidis is used in combination with galTK from Helicobacter pylori and Chepa from Campylobacter hepaticus to produce LST-a starting from lactose as acceptor saccharide.

In one example, LgtA from Neisseria meningitidis is used in combination with galTK from Helicobacter pylori and Clari1 from Campylobacter clari to produce LST-a starting from lactose as initial substrate.

In one example, galTK from Helicobacter pylori is used in combination with Cjej1 from Campylobacter jejuni to produce LST-a starting from LNT-II as initial substrate.

In one example, galTK from Helicobacter pylori is used in combination with Ccol2 from Campylobacter coli to produce LST-a starting from LNT-1l as initial substrate.

In one example, galTK from Helicobacter pylori is used in combination with Csub1 from Campylobacter subantarcticus to produce LST-a starting from LNT-II as initial substrate.

In one example, galTK from Helicobacter pylori is used in combination with Chepa from Campylobacter hepaticus to produce LST-a starting from LNT-II as initial substrate.

In one example, galTK from Helicobacter pylori is used in combination with Clari1 from Campylobacter clari to produce LST-a starting from LNT-II as initial substrate.

### Glycosyl-Donor—Nucleotide-Activated Sugar Pathways

When carrying out the method of this disclosure, preferably a glycosyltransferase mediated glycosylation reaction takes place in which an activated sugar nucleotide serves as glycosyl-donor. An activated sugar nucleotide generally has a phosphorylated glycosyl residue attached to a nucleoside. A specific glycosyl transferase enzyme accepts only a specific sugar nucleotide. Thus, preferably the following activated sugar nucleotides are involved in the glycosyl transfer: glucose-UDP-GlcNAc, UDP-galactose, UDP-glucose, UDP-N-acetylglucosamine, UDP-N-acetylgalactosamine (GlcNAc) and CMP-N-acetylneuraminic acid. The genetically modified cell according to the present disclosure can comprise one or more pathways to produce a nucleotide-activated sugar selected from the group consisting of glucose-UDP-GlcNAc, GDP-fucose, UDP-galactose, UDP-glucose, UDP-N-acetylglucosamine, UDP-N-acetylgalactosamine and CMP-N-acetylneuraminic acid.

In one embodiment of the method, the genetically modified cell is capable of producing one or more activated sugar nucleotides mentioned above by a de novo pathway. In this regard, an activated sugar nucleotide is made by the cell under the action of enzymes involved in the de novo biosynthetic pathway of that respective sugar nucleotide in a stepwise reaction sequence starting from a simple carbon source like glycerol, sucrose, fructose or glucose (for a review for monosaccharide metabolism see e.g. H. H. Freeze and A. D. Elbein: Chapter 4: Glycosylation precursors, in: Essentials of Glycobiology, 2nd edition (Eds. A. Varki et al.), Cold Spring Harbour Laboratory Press (2009).

The enzymes involved in the de novo biosynthetic pathway of an activated sugar nucleotide can be naturally present in the cell or introduced into the cell by means of gene technology or recombinant DNA techniques, all of them are parts of the general knowledge of the skilled person.

In another embodiment, the genetically modified cell can utilize salvaged monosaccharides for sugar nucleotide. In the salvage pathway, monosaccharides derived from degraded oligosaccharides are phosphorylated by kinases, and converted to nucleotide sugars by pyrophosphorylases. The enzymes involved in the procedure can be heterologous ones, or native ones of the host cell.

### Sialic Acid Sugar Nucleotide Synthesis Pathway

Preferably, the genetically modified cell according to the present disclosure comprises a sialic acid sugar nucleotide synthesis capability, i.e., the genetically modified cell comprises a biosynthetic pathway for making a sialate sugar nucleotide, such as CMP-N-acetylneuraminic acid as glycosyl-donor for the alpha-2,3-sialyltransferase of the present disclosure. E.g., the genetically modified cell comprises a sialic acid synthetic capability through provision of an exogenous UDP-GlcNAc 2-epimerase (e.g., neuC of Campylobacter jejuni (GenBank AAK91727.1) or equivalent (e.g., (GenBank CAR04561.1), a Neu5Ac synthase (e.g., neuB of C. jejuni(GenBank AAK91726.1) or equivalent, (e.g., Flavobacterium limnosediminis sialic acid synthase, GenBank WP_023580510.1), and/or a CMP-Neu5Ac synthetase (e.g., neuA of C. jejuni (GenBank AAK91728.1) or equivalent, (e.g., Vibrio brasiliensis CMP-sialic acid synthase, GenBank WP_006881452.1).

In one or more examples UDP-GlcNAc 2-epimerase, CMP-Neu5Ac synthetase, Neu5Ac synthase from Campylobacter jejuni, also referred to as neuBCA from Campylobacter jejuni or simply the neuBCA operon, may be plasmid borne or integrated into the genome of the genetically modified cell. Preferably, the sialic acid sugar nucleotide pathway is encoded by the nucleic acid sequence encoding neuBCA from Campylobacter jejuni (SEQ ID NO: 38) or a functional variant thereof having an amino acid sequence which is at least 80% identical, such as at least 85%, such as at least 90% or such as at least 99% to SEQ ID NO: 38.

Additionally, the nucleic acid sequence encoding neuBCA is preferably encoded from a high-copy plasmid bearing the neuBCA operon. In embodiments, the high-copy plasmid is the BlueScribe M13 plasmid (pBS). In relation to the present disclosure, a high-copy plasmid is a plasmid that that replicates to a copy number above 50 when introduced into the cell.

### A Deficient Sialic Acid Catabolic Pathway

The genetically modified cell of the present disclosure preferably has a deficient sialic acid catabolic pathway. By “sialic acid catabolic pathway” is meant a sequence of reactions, usually controlled, and catalysed by enzymes, which results in the degradation of sialic acid. An exemplary sialic acid catabolic pathway described hereafter is the E. coli pathway. In this pathway, sialic acid (Neu5Ac; N-acetylneuraminic acid) is degraded by the enzymes NanA (N-acetylneuraminic acid lyase) and NanK (N-acetylmannosamine kinase) and NanE (N-acetylmannosamine-6-phosphate epimerase), all encoded from the nanATEK-yhcH operon, and repressed by NanR (http://ecocyc.org/ECOLI). A deficient sialic acid catabolic pathway is rendered in the E. coli host by introducing a mutation in the endogenous nanA (N-acetylneuraminate lyase) (e.g., GenBank Accession Number D00067.1(GL216588)) and/or nanK (N-acetylmannosamine kinase) genes (e.g., GenBank Accession Number (amino acid) BAE77265.1 (GL85676015)), and/or nanE (N-acetylmannosamine-6-phosphate epimerase, GI: 947745), incorporated herein by reference). Optionally, the nanT (N-acetylneuraminate transporter) gene is also inactivated or mutated. Other intermediates of sialic acid metabolism include: (ManNAc-6-P) N-acetylmannosamine-6-phosphate; (GlcNAc-6-P) N-acetylglucosamine-6-phosphate; (GlcN-6-P) Glucosamine-6-phosphate, and (Fruc-6-P) Fructose-6-phosphate. In some preferred embodiments, nanA is mutated. In other preferred embodiments, nanA and nanKare mutated, while nanE remains functional. In another preferred embodiment, nanA and nanE are mutated, while nanK has not been mutated, inactivated or deleted. A mutation is one or more changes in the nucleic acid sequence coding the gene product of nanA, nanK, nanE, and/or nanT. E.g., the mutation may be 1, 2, up to 5, up to 10, up to 25, up to 50 or up to 100 changes in the nucleic acid sequence. E.g., the nanA, nanK, nanE, and/or nanT genes are mutated by a null mutation. Null mutations as described herein encompass amino acid substitutions, additions, deletions, or insertions, which either cause a loss of function of the enzyme (i.e., reduced or no activity) or loss of the enzyme (i.e., no gene product). By “deleted” is meant that the coding region is removed completely or in part such that no (functional) gene product is produced. By inactivated is meant that the coding sequence has been altered such that the resulting gene product is functionally inactive or encodes for a gene product with less than 100%, e.g., 90%, 80%, 70%, 60%, 50%, 40%, 30% or 20% of the activity of the native, naturally occurring, endogenous gene product. Thus, in the present disclosure, nanA, nanK, nanE, and/or nanT genes are preferably inactivated.

### Major Facilitator Superfamily (MFS) Transporter Proteins

The oligosaccharide product, such as the HMO produced by the cell, can be accumulated both in the intra- and the extracellular matrix. The product can be transported to the supernatant in a passive way, i.e., it diffuses outside across the cell membrane. The more complex HMO products may remain in the cell, which is likely to eventually impair cellular growth, thereby affecting the possible total yield of the product from a single fermentation. The HMO transport can be facilitated by major facilitator superfamily transporter proteins that promote the effluence of sugar derivatives from the cell to the supernatant. The major facilitator superfamily transporter can be present exogenously or endogenously and is overexpressed under the conditions of the fermentation to enhance the export of the oligosaccharide derivative (HMO) produced. The specificity towards the sugar moiety of the product to be secreted can be altered by mutation by means of known recombinant DNA techniques.

Thus, the genetically modified cell according to the present invention can further comprise a nucleic acid sequence encoding a major facilitator superfamily transporter protein capable of exporting the sialylated human milk oligosaccharide product or products.

In the resent years, several new and efficient major facilitator superfamily transporter proteins have been identified, each having specificity for different recombinantly produced HMOs and development of recombinant cells expressing said proteins are advantageous for high scale industrial HMO manufacturing. WO2021/123113 claim different E. coli and heterologous transporters for the export of 3′SL, 6′SL and LST-a.

Thus, in one or more exemplary embodiments, the genetically engineered cell according to the method described herein further comprises a gene product that acts as a major facilitator superfamily transporter. The gene product that acts as a major facilitator superfamily transporter may be encoded by a recombinant nucleic acid sequence that is expressed in the genetically engineered cell. The recombinant nucleic acid sequence encoding a major facilitator superfamily transporter, may be integrated into the genome of the genetically engineered cell, or expressed using a plasmid.

In one embodiment, the genetically modified cell described herein comprises a nucleic acid sequence encoding a major facilitator superfamily transporter protein capable of exporting the sialylated human milk oligosaccharide product into the extracellular medium, in particular, the transporters with specificity towards LST-a and/or 3′SL are preferred.

### Nec

In one embodiment, the genetically modified cell described herein comprises a nucleic acid sequence encoding an efflux transporter protein capable of exporting the sialylated human milk oligosaccharide product, such as 3′SL and/or LST-a; into the extracellular medium. In the current context, said efflux transporter protein is preferably a heterologous gene encoding a putative MFS (major facilitator superfamily) transporter protein, originating from the bacterium Rosenbergiella nectarea. More specifically, the disclosure relates to a genetically modified cell optimized to produce an oligosaccharide, in particular a sialylated HMO, comprising a recombinant nucleic acid encoding a protein having at least 80%, such as at least 85%, such as at least 90% such as at least 95% or 100% sequence identity to the amino acid sequence of the amino acid sequence having GenBank accession ID WP_092672081.1.

Additionally, the MFS transporter protein with the GenBank accession ID WP_092672081.1 is further described in WO2021/148615 and is identified herein as “NEC protein” or “NEC transporter” or “NEC”, interchangeably; a nucleic acid sequence encoding Nec protein is identified herein as “nec coding nucleic acid/DNA” or “nec gene” or “nec”.

Nec is expected to facilitate an increase in the efflux of the produced sialylated HMOs, e.g., 3′SL in the genetically engineered cells of the current disclosure.

### Fred/YberC

In embodiments, the genetically modified cell of the present disclosure comprises a nucleic acid sequence encoding an efflux transporter protein capable of exporting the simple sialylated human milk oligosaccharide product such as 3′SL and 6′SL into the extracellular medium. In the current context, said efflux transporter protein is preferably a heterologous gene encoding a putative MFS (major facilitator superfamily) transporter protein, originating from the bacterium Yersinia frederiksenii and/or the bacterium Yersinia bercovieri. More specifically, the disclosure relates to a genetically modified cell optimized to produce an oligosaccharide, in particular a sialylated HMO, comprising a recombinant nucleic acid encoding a protein having at least 80%, such as at least 85%, such as at least 90% such as at least 95% or 100% sequence identity to the amino acid sequence of the amino acid sequence having the GenBank accession ID WP_087817556.1 or GenBank accession EEQ08298.

The MFS transporter protein with the GenBank accession ID WP_087817556.1 is further described in WO2021148620 and is identified herein as “Fred protein” or “Fred transporter” or “Fred”, interchangeably; a nucleic acid sequence encoding Fred protein is identified herein as “fred coding nucleic acid/DNA” or “fred gene” or “fred”.

Additionally, the MFS transporter protein with the GenBank accession ID EEQ08298 is further described in WO2021148610 and is identified herein as “YberC protein” or “YberC transporter” or “YberC”, interchangeably; a nucleic acid sequence encoding YberC protein is identified herein as “yberC coding nucleic acid/DNA” or “yberC gene” or “yberC”.

Fred and YberC facilitate an increase in the efflux of the produced sialylated HMOs, e.g., 3′SL in the genetically engineered cells of the current disclosure.

### Substrate Importers

Conventionally HMO production is performed with lactose as the initial substrate which the cell is capable of importing e.g., via a lactose permease, such as LacY. It may be beneficial to overexpress the lactose permease, e.g., by exchanging the native promoter with a stronger promoter or by inserting a (additional) copy of the lactose permease into the genome of the host cell.

In alternative embodiments the initial substrate may be lacto-N-triose (LNT-II) or lacto-N-tetraose (LNT). For the production of LST-a this will result in a reduced amount of bi-product since 3′SL and potentially LNT-II will not be produced by such a cell.

In alternative embodiments the genetically modified cell is capable of importing LNT-II or LNT as the initial substrate for the formation of LST-a. To enable this, the genetically modified cell is additionally modified such that it comprises at least one recombinant nucleic acid sequence and/or a cluster of recombinant nucleic acid sequences encoding a transporter protein and/or a cluster of proteins capable of importing the initial substrate.

PCT/EP2022/084101 describes potential importers of trisaccharides, tetrasaccharides and pentasaccharides (acceptor oligosaccharides/HMO precursor molecules) of relevance in the HMO production, e.g., 2-fucosyllactose (2′FL), 3-fucosyllactose (3FL), lacto-N-triose (LNT-II), lacto-N-tetraose (LNT), lacto-N-neotetraose (LNnT) and lacto-N-fucopentose I (LNFP-I).

In embodiments of the present disclosure the genetically modified cell may comprise a recombinant nucleic acid sequence encoding a transporter protein capable of importing an intermediate (acceptor) oligosaccharide of at least three monosaccharide units, such as LNT-II, wherein said transporter protein is a mutated lactose permease (LacY) as shown in table 7.

TABLE 7
List of exemplary mutants of the E. coli DH1
K12 lactose permease LacY (SEQ ID NO: 56) that
could be useful for the import of LNT-II

| | | | Wild-type | Mutant |
|---|---|---|---|---|
| Name | Mutation | Position | amino acid | amino acid |
| mut1 | Y236N | 236 | Tyr | Asn |
| mut2 | Y236H | 236 | Tyr | His |
| mut3 | S306T | 306 | Ser | Thr |
| mut4 | A177V | 177 | Ala | Val |
| mut5 | H322N | 322 | His | Asn |
| Mut6 | I303F | 303 | Ile | Phe |
| mut6 | Y236H, | 236 | Tyr | His |
| | S306T | 306 | Ser | Thr |
| mut7 | A177V, | 177 | Ala | Val |
| | Y236H | 236 | Tyr | His |
| mut8 | A177V, | 177 | Ala | Val |
| | I303F | 303 | Ile | Phe |
| mut9 | A177V, | 177 | Ala | Val |
| | H322N | 322 | His | Asn |
| mut10 | A177V, | 177 | Ala | Val |
| | S306T | 306 | Ser | Thr |
| mut11 | A177V, | 177 | Ala | Val |
| | Y236N, | 236 | Tyr | Asn |
| | S306T | 306 | Ser | Thr |

In preferred embodiments the genetically modified cell expresses a mutated lactose permease selected from a lactose permease of SEQ ID NO: 56 or a lactose permease with 90% identity to SEQ ID NO: 56, wherein said lactose permease has one or more mutations selected from the group consisting of Y236N, Y236H, S306T, A177V, H322N, 303F, Y236H+S306T, 177V+Y236H, A177V+I303F, A177V+H322N, A177V+S306T or A177V+Y236N+S306T and wherein the mutation is at the corresponding position in SEQ ID NO: 56.

In other embodiments of the present disclosure the genetically modified cell may comprise a recombinant nucleic acid sequence encoding a transporter protein capable of importing an intermediate (acceptor) oligosaccharide of at least three monosaccharide units, such as LNT-II or of at least four monosaccharide units, such as LNT, into said cell, wherein said transporter protein is a MFS-transporter protein of gram-positive origin or an ABC-transporter protein cluster of gram-positive origin capable of importing an acceptor oligosaccharide of at least three or four monosaccharide units into a cell as shown in table 8.

TABLE 8
ABC- and MFS-transporters from gram-positive bacteria with an indication
of the precursor oligosaccharide the transporter is expected to import.
The ABC transporters are composed of three to four genes. For ease of
reference each transporter has been given a transporter ID (TP ID)

| | | | | Expected | |
|---|---|---|---|---|---|
| TP | TP | | | oligosaccharide | |
| ID | type | Gene Name | GenBank ID | transport | Origin |
| 1 | MFS | Blon_0247 | ACJ51375.1 | LNnT | B. infantis ATCC |
| | | | | | 15697 = JCM 1222 |
| 2 | MFS | Blon_0431 | ACJ51548.1 | oligosaccharide | B. infantis ATCC |
| | | | | | 15697 = JCM 1222 |
| 3 | MFS | Blon_0788 | ACJ51891.1 | oligosaccharide | B. infantis ATCC |
| | | | | | 15697 = JCM 1222 |
| 13 | MFS | Blon_0962 | ACJ52061.1 | LNT-II, | B. infantis ATCC |
| | | | | Fucosylated- | 15697 = JCM 1222 |
| | | | | HMO | |
| 14 | MFS | Blon_2307 | ACJ53365.1 | Fucosylated- | B. infantis ATCC |
| | | | | HMO | 15697 = JCM 1222 |
| 4 | MFS | Blon_2400 | ACJ53455.1 | oligosaccharide | B. infantis ATCC |
| | | | | | 15697 = JCM 1222 |
| 5 | ABC | Blon_2341 | ACJ53399.1 | LN(n)T | B. infantis ATCC |
| | | | | | 15697 = JCM 1222 |
| | | Blon_2342 | ACJ53400.1 | | B. infantis ATCC |
| | | | | | 15697 = JCM 1222 |
| | | Blon_2343 | ACJ53401.1 | | B. infantis ATCC |
| | | | | | 15697 = JCM 1222 |
| | | Blon_2344 | ACJ53402.1 | | B. infantis ATCC |
| | | | | | 15697 = JCM 1222 |
| 6 | ABC | Blon_2345 | ACJ53403.1 | LN(n)T | B. infantis ATCC |
| | | | | | 15697 = JCM 1222 |
| | | Blon_2346 | ACJ53404.1 | | B. infantis ATCC |
| | | | | | 15697 = JCM 1222 |
| | | Blon_2347 | ACJ53405.1 | | B. infantis ATCC |
| | | | | | 15697 = JCM 1222 |
| 15 | ABC | Blon_0341/ | ACJ51465.1/ | 2′-FL, 3-FL, | B. infantis ATCC |
| | | Blon_2204 | ACJ53264.1 | DFL, LNFP-I | 15697 = JCM 1222 |
| | | Blon_0342/ | ACJ51466.1/ | | B. infantis ATCC |
| | | Blon_2203 | ACJ53263.1 | | 15697 = JCM 1222 |
| | | Blon_2202 | ACJ53262.1 | | B. infantis ATCC |
| | | | | | 15697 = JCM 1222 |
| 16 | ABC | Blon_0341/ | ACJ51465.1/ | 2′-FL, 3-FL, | B. infantis ATCC |
| | | Blon_2204 | ACJ53264.1 | DFL, LNFP-I | 15697 = JCM 1222 |
| | | Blon_0342/ | ACJ51466.1/ | | B. infantis ATCC |
| | | Blon_2203 | ACJ53263.1 | | 15697 = JCM 1222 |
| | | Blon_0343 | ACJ51467.1 | | B. infantis ATCC |
| | | | | | 15697 = JCM 1222 |
| 7 | ABC | Blon_0883 | ACJ51983.1 | LN(n)T, Lewis a, | B. infantis ATCC |
| | | | | type 1 H- | 15697 = JCM 1222 |
| | | Blon_0884 | ACJ51984.1 | trisaccharide | B. infantis ATCC |
| | | | | | 15697 = JCM 1222 |
| | | Blon_0885 | ACJ51985.1 | | B. infantis ATCC |
| | | | | | 15697 = JCM 1222 |
| | | Blon_0886 | ACJ51986.1 | | B. infantis ATCC |
| | | | | | 15697 = JCM 1222 |
| 8 | ABC | Blon_2177 | ACJ53238.1 | LNB, LN(n)T, | B. infantis ATCC |
| | | | | LNO, LNH, sialyl | 15697 = JCM 1222 |
| | | Blon_2176 | ACJ53237.1 | LNT | B. infantis ATCC |
| | | | | | 15697 = JCM 1222 |
| | | Blon_2175 | ACJ53236.1 | | B. infantis ATCC |
| | | | | | 15697 = JCM 1222 |
| 9 | ABC | RHOM_04095 | AEN95941.1 | LNT | R. hominis A2-183 |
| | | RHOM_04100 | AEN95942.1 | | R. hominis A2-183 |
| | | RHOM_04105 | AEN95943.1 | | R. hominis A2-183 |
| 10 | ABC | HMPREF0373_{—} | ERK42291.1 | LNT | E. ramulus ATCC |
| | | 02960 | | | 29099 |
| | | HMPREF0373_{—} | ERK42292.1 | | E. ramulus ATCC |
| | | 02961 | | | 29099 |
| | | HMPREF0373_{—} | ERK42293.1 | | E. ramulus ATCC |
| | | 02962 | | | 29099 |
| 17 | ABC | BBKW_1838/ | BAQ29973.1/ | 2-′FL, 3-FL, | B. kashiwanohense |
| | | BKAS_1807 | KFI63925.1 | DFL, LNFP-I | JCM 15439 |
| | | BBKW_1839/ | BAQ29974.1/ | (FL-2) | B. kashiwanohense |
| | | BKAS_1806 | KFI63924.1 | | JCM 15439 |
| | | BBKW_1840/ | BAQ29975.1/ | | B. kashiwanohense |
| | | BKAS_1805 | KFI63923.1 | | JCM 15439 |
| 18 | ABC | BBPC_1775 | BAR04453.1 | 2′-FL, 3-FL, | B. pseudocatenulatum |
| | | | | LNT-II, DFL, | JCM 1200 |
| | | BBPC_1776 | BAR04454.1 | LNFP-I | B. pseudocatenulatum |
| | | | | | JCM 1200 |
| | | BBPC_1777 | BAR04455.1 | | B. pseudocatenulatum |
| | | | | | JCM 1200 |
| 11 | ABC | BBR_0527/IntP1 | ABE95224.1 | LNT-II, LN(n)T | B. breve UCC2003 |
| | | BBR_0528/IntP2 | ABE95225.1 | | B. breve UCC2003 |
| | | BBR_0530/IntS | ABE95226.1 | | B. breve UCC2003 |
| | | BBR_0531 | ABE95228.1 | | B. breve UCC2003 |
| 12 | ABC | BBR_1554/nahS | ABE96225.1 | LNnT | B. breve UCC2003 |
| | | BBR_1558 | ABE96228.1 | | B. breve UCC2003 |
| | | BBR_1559 | ABE96229.1 | | B. breve UCC2003 |
| | | BBR_1560 | ABE96230.1 | | B. breve UCC2003 |

Typically, the genetically modified cell lacks enzymatic activity liable to degrade the acceptor oligosaccharide of at least three, four or five monosaccharide units imported into the cell.

In embodiments the genetically modified cell comprises an MFS transporter or an ABC transporter disclosed in table 8. More preferably the genetically modified cell comprises an MFS transporter or an ABC transporter selected from the group consisting of a) Blon_0962 (TP ID: 13 in table 8) or an amino acid sequence with at least 80%, such as 85%, such as 90%, such as 95% identity to Genbank accession nr. ACJ52061.1; b) BBPC_1775, 1776, 1777 (TP ID:18 in table 8) comprising three sub-units with the amino acid sequences comprising or consisting of Genbank accession nrs BAR04453.1, BAR04454.1 and BAR04455.1 or amino acid sequences with at least 80%, such as 85%, such as 90%, such as 95% identity to Genbank accession nrs BAR04453.1, BAR04454.1 and BAR04455.1 and c) Bbr_0527, 0528, 0530, 0531 (TP ID: 11 in table 8). comprising four sub-units with the amino acid sequences comprising or consisting of Genbank accession nrs ABE95224.1, ABE95225.1, ABE95226.1 and ABE95228 or amino acid sequences with at least 80%, such as 85%, such as 90%, such as 95% identity to Genbank accession nrs ABE95224.1, ABE95225.1, ABE95226.1 and ABE95228.

### HMO Concentrations

The genetically modified cell comprising more than one glycosyltransferase described herein will generally produce a mixture of HMOs as a result of the multistep process towards the final HMO product. In the production of LST-a from lactose as the initial substrate, it is expected that 3′SL (sialylated lactose), LNT-II, LNT and LST-a are present at the end of the cultivation.

The HMO products produced by the methods disclosed herein can be described by their ratios in a mixture of HMOs. The “ratio” as described herein is understood as the ratio between two amounts of HMOs, such as, but not limited to, the amount of one HMO divided by the amount of the other HMO, or the amount of one HMO divided by the total amount of HMOs.

In one embodiment, following cultivation of the genetically modified cell as described herein, the mixture of HMOs has a molar % of LST-a between 8.5% to 30% and 3′SL between 4.5% to 25%, such as molar % of LST-a between 10% to 26% and 3′SL between 5% to 20%. In a preferred embodiment, the molar % of LST-a is above 9%, such as above 15%, such as above 18%, such as above 25% of the total HMO. In another preferred embodiment, the molar % of 3′SL is below 30%, such as below 25%, such as below 20%, such as below 15%, such as below 10% of the total HMO.

The molar % ratios supported by experimental data from the Examples shows exemplary HMO composition ranges, wherein the ratio of LST-a:3′SL is in the range from 1:2 to 2:1. In a preferred embodiment, the LST-a:3′SL ratio is at least 2:1, preferably with an even higher LST-a content than 3′SL content, e.g. 3:1 or 4:1, as observed in the fermentations in Example 2.

In some embodiments, the genetically modified cell of the present disclosure expresses Ccol2 comprising or consisting of the amino acid sequence of SEQ ID NO: 1 or an amino acid sequence with at least 80%, such as at least 85%, such as at least 90%, such as at least 95%, or such as at least 99% identity to SEQ ID NO: 1, and the molar % content of LST-a produced by the genetically modified cell is above 20%, such as above 25%, such as above 30% of the total HMO.

In some embodiments, the genetically modified cell of the present disclosure expresses Ccol2 comprising or consisting of the amino acid sequence of SEQ ID NO: 1 or an amino acid sequence with at least 80%, such as at least 85%, such as at least 90%, such as at least 95%, or such as at least 99% identity to SEQ ID NO: 1, and the molar % content of 3′SL produced by the genetically modified cell is below 20%, such as below 15%, such as below 10% of the total HMO.

In some embodiments, the genetically modified cell of the present disclosure expresses Ccol2 comprising or consisting of the amino acid sequence of SEQ ID NO: 1 or an amino acid sequence with at least 80%, such as at least 85%, such as at least 90%, such as at least 95%, or such as at least 99% identity to SEQ ID NO: 1, and the ratio of LST-a:3′SL is above 2:1, i.e. the genetically modified cell produce more than 22% LST-a and less than 11% 3′SL.

In some embodiments, the genetically modified cell of the present disclosure expresses Cjej1 comprising or consisting of the amino acid sequence of SEQ ID NO: 2 or an amino acid sequence with at least 80%, such as at least 85%, such as at least 90%, such as at least 95%, or such as at least 99% identity to SEQ ID NO: 2, and the molar % content of LST-a produced by the genetically modified cell is above 15%, such as above 20%, such as above 25% of the total HMO.

In some embodiments, the genetically modified cell of the present disclosure expresses Cjej1 comprising or consisting of the amino acid sequence of SEQ ID NO: 2 or an amino acid sequence with at least 80%, such as at least 85%, such as at least 90%, such as at least 95%, or such as at least 99% identity to SEQ ID NO: 2, and the molar % content of 3′SL produced by the genetically modified cell is below 20%, such as below 15%, such as below 10% of the total HMO.

In some embodiments, the genetically modified cell of the present disclosure expresses Cjej1 comprising or consisting of the amino acid sequence of SEQ ID NO: 2 or an amino acid sequence with at least 80%, such as at least 85%, such as at least 90%, such as at least 95%, or such as at least 99% identity to SEQ ID NO: 2, and the ratio of LST-a:3′SL above 1.5:1. i.e. the genetically modified cell produce more than 18% LST-a and less than 10% 3′SL.

In some embodiments, the genetically modified cell of the present disclosure expresses Csub1 comprising or consisting of the amino acid sequence of SEQ ID NO: 3 or an amino acid sequence with at least 80%, such as at least 85%, such as at least 90%, such as at least 95%, or such as at least 99% identity to SEQ ID NO: 3, and the molar % content of LST-a produced by the genetically modified cell is above 10% of the total HMO.

In some embodiments, the genetically modified cell of the present disclosure expresses Csub1 comprising or consisting of the amino acid sequence of SEQ ID NO: 3 or an amino acid sequence with at least 80%, such as at least 85%, such as at least 90%, such as at least 95%, or such as at least 99% identity to SEQ ID NO: 3, and the molar % content of 3′SL produced by the genetically modified cell is below 20%, such as below 15%, such as below 10% of the total HMO.

In some embodiments, the genetically modified cell of the present disclosure expresses Csub1 comprising or consisting of the amino acid sequence of SEQ ID NO: 3 or an amino acid sequence with at least 80%, such as at least 85%, such as at least 90%, such as at least 95%, or such as 99% identity to SEQ ID NO: 3, and the ratio of LST-a:3′SL above 2:1, i.e. the genetically modified cell produce more than 10% LST-a and less than 5% 3′SL.

In some embodiments, the genetically modified cell of the present disclosure expresses Chepa comprising or consisting of the amino acid sequence of SEQ ID NO: 4 or an amino acid sequence with at least 80%, such as at least 85%, such as at least 90%, such as at least 95%, or such as at least 99% identity to SEQ ID NO: 4, and the molar % content of LST-a produced by the genetically modified cell is above 10% of the total HMO.

In some embodiments, the genetically modified cell of the present disclosure expresses Chepa comprising or consisting of the amino acid sequence of SEQ ID NO: 4 or an amino acid sequence with at least 80%, such as at least 85%, such as at least 90%, such as at least 95%, or such as at least 99% identity to SEQ ID NO: 4, and the molar % content of 3′SL produced by the genetically modified cell is below 20%, such as below 15%, such as below 10% of the total HMO.

In some embodiments, the genetically modified cell of the present disclosure expresses Clari1 comprising or consisting of the amino acid sequence of SEQ ID NO: 4 or an amino acid sequence with at least 80%, such as at least 85%, such as at least 90%, such as at least 95%, or such as at least 99% identity to SEQ ID NO: 4, and the molar % content of LST-a produced by the genetically modified cell is above 9% of the total HMO.

In some embodiments, the genetically modified cell of the present disclosure expresses Clari1 comprising or consisting of the amino acid sequence of SEQ ID NO: 5 or an amino acid sequence with at least 80%, such as at least 85%, such as at least 90%, such as at least 95%, or such as at least 99% identity to SEQ ID NO: 5, and the molar % content of 3′SL produced by the genetically modified cell is below 20%, such as below 15%, such as below 10% of the total HMO.

### The Genetically Modified Cell

In the present context, the terms “a genetically modified cell” and “a genetically engineered cell” are used interchangeably. As used herein “a genetically modified cell” is a host cell whose genetic material has been altered by human intervention using a genetic engineering technique, such a technique is e.g., but not limited to transformation or transfection e.g., with a heterologous polynucleotide sequence, Crisper/Cas editing and/or random mutagenesis. In one embodiment the genetically engineered cell has been transformed or transfected with a recombinant nucleic acid sequence.

The genetic modifications can e.g., be selected from inclusion of glycosyltransferases, and/or metabolic pathway engineering and/or inclusion of MFS transporters and/or inclusion of substrate importers as described in the above sections, which the skilled person will know how to combine into a genetically modified cell capable of producing one or more sialylated HMO's.

In one aspect of the disclosure, the genetically modified cell comprises a recombinant nucleic acid sequence encoding an enzyme with α-2,3-sialyltransferase activity, which is capable of producing at least 9% LST-a of the total molar HMO content produced by the cell.

In one embodiment the genetically modified cell capable of producing a sialylated HMO, comprises a recombinant nucleic acid sequence encoding an enzyme with α-2,3-sialyltransferase activity, wherein said enzyme is selected from the group consisting of:
a. Ccol2 comprising or consisting of the amino acid sequence of SEQ ID NO: 1 or an amino acid sequence with at least 80% identity to SEQ ID NO: 1,
b. Cjej1 comprising or consisting of the amino acid sequence of SEQ ID NO: 2 or an amino acid sequence with at least 80% identity to SEQ ID NO: 2,
c. Csub1 comprising or consisting of the amino acid sequence of SEQ ID NO: 3 or an amino acid sequence with at least 80% identity to SEQ ID NO: 3,
d. Chepa comprising or consisting of the amino acid sequence of SEQ ID NO: 4 or an amino acid sequence with at least 80% identity to SEQ ID NO: 4, and
e. Clari1 comprising or consisting of the amino acid sequence of SEQ ID NO: 5 or an amino acid sequence with at least 80% identity to SEQ ID NO: 5.

In a presently preferred embodiment, the genetically modified cell capable of producing a sialylated HMO, which comprises a recombinant nucleic acid sequence encoding an enzyme with α-2,3-sialyltransferase activity as described herein is capable of producing LST-a in an amount of at least 9% of the total molar HMO content produced by the cell.

The genetically engineered cell is preferably a microbial cell, such as a prokaryotic cell or eukaryotic cell. Appropriate microbial cells that may function as a host cell include bacterial cells, archaebacterial cells, algae cells and fungal cells.

The genetically engineered cell may be e.g., a bacterial or yeast cell. In one preferred embodiment, the genetically engineered cell is a bacterial cell.

### Host Cells

Regarding the host cells, there are, in principle, no limitations; they may be eubacteria (gram-positive or gram-negative) or archaebacteria or fungi or even mammalian cells, as long as they allow genetic manipulation for insertion of a gene of interest and can be cultivated on a manufacturing scale. Preferably, the host cell has the property to allow cultivation to high cell densities.

In embodiments, the genetically engineered cell is a microorganism. The genetically engineered cell is preferably a microbial cell, such as a prokaryotic cell or eukaryotic cell. Appropriate microbial cells that may function as a host cell include bacterial cells, archaebacterial cells, algae cells and fungal cells.

The genetically engineered cell may be e.g., a bacterial or yeast cell. In one preferred embodiment, the genetically engineered cell is a bacterial cell.

Non-limiting examples of bacterial host cells that are suitable for recombinant industrial production of an HMO(s) according to the disclosure could be Erwinia herbicola (Pantoea agglomerans), Citrobacter freundii, Campylobacter sp, Pantoea citrea, Pectobacterium carotovorum, or Xanthomonas campestris. Bacteria of the genus Bacillus may also be used, including Bacillus subtilis, Bacillus licheniformis, Bacillus coagulans, Bacillus thermophilus, Bacillus laterosporus, Bacillus megaterium, Bacillus mycoides, Bacillus pumilus, Bacillus lentus, Bacillus cereus, and Bacillus circulans. Similarly, bacteria of the genera Lactobacillus and Lactococcus may be engineered using the methods of this disclosure, including but not limited to Lactobacillus acidophilus, Lactobacillus salivarius, Lactobacillus plantarum, Lactobacillus helveticus, Lactobacillus delbrueckii, Lactobacillus rhamnosus, Lactobacillus bulgaricus, Lactobacillus crispatus, Lactobacillus gasseri, Lactobacillus casei, Lactobacillus reuteri, Lactobacillus jensenii, and Lactococcus lactis. Streptococcus thermophiles and Proprionibacterium freudenreichii are also suitable bacterial species. Also included as part of this disclosure are strains, engineered as described here, from the genera Enterococcus (e.g., Enterococcus faecium and Enterococcus thermophiles), Bifidobacterium (e.g., Bifidobacterium longum, Bifidobacterium infantis, and Bifidobacterium bifidum), Streptomyces spp., Sporolactobacillus spp., Micromomospora spp., Micrococcus spp., Rhodococcus spp., and Pseudomonas (e.g., Pseudomonas fluorescens and Pseudomonas aeruginosa).

Non-limiting examples of fungal host cells that are suitable for recombinant industrial production of a heterologous product are e.g., yeast cells, such as Komagataella phaffii, Kluyveromyces lactis, Yarrowia lipolytica, Pichia pastoris, and Saccaromyces cerevisiae or filamentous fungi such as Aspargillus sp, Fusarium sp or Thricoderma sp, exemplary species are A. niger, A. nidulans, A. oryzae, F. solani, F. graminearum and T. reesei.

In one or more exemplary embodiments, the genetically engineered cell is selected from the group consisting of Escherichia coli, Corynebacterium glutamicum, lactobacillus lactis, Bacillus subtilis, Streptomyces lividans, Yarrowia lipolytica, Pichia pastoris and Saccharomyces cerevisiae.

In one or more exemplary embodiments, the genetically engineered cell is selected from the group consisting of of Escherichia Coli, Bacillus subtilis, lactobacillus lactis, Corynebacterium glutamicum, Yarrowia lipolytica, Pichia pastoris, and Saccharomyces cerevisiae.

In one or more exemplary embodiments, the genetically engineered cell is B. subtilis.

In one or more exemplary embodiments, the genetically engineered cell is S. Cerevisiae or P pastoris.

In one or more exemplary embodiments, the genetically engineered cell is Corynebacterium glutamicum.

In one or more exemplary embodiments, the genetically engineered cell is Escherichia coli.

In one or more exemplary embodiments, the disclosure relates to a genetically engineered cell, wherein the cell is derived from the E. coli K-12 strain or DE3.

### A Recombinant Nucleic Acid Sequence

The present disclosure relates to a genetically modified cell comprising a recombinant nucleic acid sequence encoding an enzyme with α-2,3-sialyltransferase activity, such as an enzyme selected from the group consisting of Ccol2, Cjej1, Csub1, Chepa and Clari1, and wherein said cell produces Human Milk Oligosaccharides (HMO). In particular a sialylated HMO, and preferably with a molar % content of LST-a above 9% of the total HMO produced.

In the present context, the term “recombinant nucleic acid sequence”, “recombinant gene/nucleic acid/nucleotide sequence/DNA encoding” or “coding nucleic acid sequence” is used interchangeably and intended to mean an artificial nucleic acid sequence (i.e. produced in vitro using standard laboratory methods for making nucleic acid sequences) that comprises a set of consecutive, non-overlapping triplets (codons) which is transcribed into mRNA and translated into a protein when under the control of the appropriate control sequences, i.e., a promoter sequence.

The boundaries of the coding sequence are generally determined by a ribosome binding site located just upstream of the open reading frame at the 5′end of the mRNA, a transcriptional start codon (AUG, GUG or UUG), and a translational stop codon (UAA, UGA or UAG). A coding sequence can include, but is not limited to, genomic DNA, cDNA, synthetic, and recombinant nucleic acid sequences.

The term “nucleic acid” includes RNA, DNA and cDNA molecules. It is understood that, as a result of the degeneracy of the genetic code, a multitude of nucleic acid sequences encoding a given protein may be produced.

The recombinant nucleic acid sequence may be a coding DNA sequence e.g., a gene, or non-coding DNA sequence e.g., a regulatory DNA, such as a promoter sequence or other non-coding regulatory sequences.

The recombinant nucleic acid sequence may in addition be heterologous. As used herein “heterologous” refers to a polypeptide, amino acid sequence, nucleic acid sequence or nucleotide sequence that is foreign to a cell or organism, i.e., to a polypeptide, amino acid sequence, nucleic acid molecule or nucleotide sequence that does not naturally occurs in said cell or organism.

The disclosure also relates to a nucleic acid construct comprising a coding nucleic sequence, i.e. recombinant DNA sequence of a gene of interest, e.g., a sialyltransferase gene, and a non-coding regulatory DNA sequence, e.g., a promoter DNA sequence, e.g., a recombinant promoter sequence derived from the promoter sequence of the lac operon or the glp operon, or a promoter sequence derived from another genomic promoter DNA sequence, or a synthetic promoter sequence, wherein the coding and promoter sequences are operably linked.

The term “operably linked” refers to a functional relationship between two or more nucleic acid (e.g., DNA) segments. It refers to the functional relationship of a transcriptional regulatory sequence to a transcribed sequence. E.g., a promoter sequence is operably linked to a coding sequence if it stimulates or modulates the transcription of the coding sequence in an appropriate host cell or other expression system.

Generally, promoter sequences that are operably linked to a transcribed sequence are physically contiguous to the transcribed sequence, i.e., they are cis-acting.

In one exemplified embodiment, the nucleic acid construct described herein may be a part of the vector DNA, in another embodiment, the construct it is an expression cassette/cartridge that is integrated in the genome of a host cell.

Accordingly, the term “nucleic acid construct” means an artificially constructed segment of nucleic acids, in particular a DNA segment, which is intended to be inserted into a target cell, e.g., a bacterial cell, to modify expression of a gene of the genome or expression of a gene/coding DNA sequence which may be included in the construct. Thus, in embodiments, the present disclosure relates to a nucleic acid construct comprising a recombinant nucleic acid sequence encoding a sialyltransferase, wherein said recombinant nucleic acid sequence is selected from the group consisting of nucleic acid sequences encoding Ccol2, Cjej1, Csub1, Chepa, and Clari1, such as SEQ ID NO: 24, 25, 26, 27 or 28, or functional variants thereof.

One embodiment is a nucleic acid construct comprising a recombinant nucleic acid sequence encoding a sialyltransferase, wherein said recombinant nucleic acid sequence is selected from the group consisting of a) Ccol2 comprising or consisting of the nucleic acid sequences of SEQ ID NO: 24 or an nucleic acid sequence with at least 80%, such as at least 85%, such as at least 90%, such as at least 95%, or such as at least 99% identity to SEQ ID NO: 24; b) Cjej1 comprising or consisting the nucleic acid sequences of SEQ ID NO: 25 or an nucleic acid sequence with at least 80%, such as at least 85%, such as at least 90%, such as at least 95%, or such as at least 99% identity to SEQ ID NO: 25; c) Csub1 comprising or consisting the nucleic acid sequence of SEQ ID NO: 26 or an nucleic acid sequence with at least 80%, such as at least 85%, such as at least 90%, such as at least 95%, or such as at least 99% identity to SEQ ID NO: 26; d) Chepa comprising or consisting the nucleic acid sequence of SEQ ID NO: 27 or an nucleic acid sequence with at least 80%, such as at least 85%, such as at least 90%, such as at least 95%, or such as at least 99% identity to SEQ ID NO: 27, and/or e) Clari1 comprising or consisting the nucleic acid sequence of SEQ ID NO: 28 or an nucleic acid sequence with at least 80%, such as at least 85%, such as at least 90%, such as at least 95%, or such as at least 99% identity to SEQ ID NO: 28. Preferably, the sialyltransf erase encoding sequence is under the control of a promoter sequence selected from promotor sequences with a nucleic acid sequence as identified in Table 2.

TABLE 2
Selected promoter sequences

| | % Activity | | | Seq ID |
|---|---|---|---|---|
| Promoter name | relative to PgIpF* | Strength | Reference | in appl. |
| PmgIB_70UTR_SD8 | 291% | high | WO2020255054 | 23 |
| PmgIB_70UTR_SD10 | 233-281% | high | WO2020255054 | 42 |
| PmgIB_54UTR | 197% | high | WO2020255054 | 41 |
| Plac_70UTR | 182-220% | high | WO2019123324 | 20 |
| PmgIB_70UTR_SD9 | 180-226% | high | WO2020255054 | 43 |
| PmgIB_70UTR_SD4 | 153%-353% | high | WO2020255054 | 22 |
| PmgIB_70UTR_SD5 | 146-152% | high | WO2020255054 | 44 |
| PgIpF_SD4 | 140-161% | high | WO2019123324 | 45 |
| PmgIB_70UTR_SD7 | 127-173% | high | WO2019123324 | 46 |
| PmgIB_70UTR | 124-234% | high | WO2020255054 | 21 |
| PgIpA_70UTR | 102-179% | high | WO2019123324 | 47 |
| PgIpT_70UTR | 102-240% | high | WO2019123324 | 48 |
| PgIpF | 100% | high | WO2019123324 | 15 |
| PgIpF_SD10 | 88-96% | high | WO2019123324 | 49 |
| PgIpF_SD5 | 82-91% | high | WO2019123324 | 50 |
| PgIpF_SD8 | 81-82% | high | WO2019123324 | 51 |
| PmgIB_16UTR | 78-171% | high | WO2019123324 | 52 |
| PgIpF_SD9 | 73-93% | middle | WO2019123324 | 53 |
| PgIpF_SD7 | 47-57% | middle | WO2019123324 | 18 |
| PgIpF_SD6 | 46-47% | middle | WO2019123324 | 54 |
| PgIpA_16UTR | 38-64% | middle | WO2019123324 | 55 |
| Plac_wt* | 15-28% | low | WO2019123324 | 19 |
| PgIpF_SD3 | 9% | low | WO2019123324 | 17 |
| PgIpF_SD1 | 5% | low | WO2019123324 | 16 |

*The promoter activity is assessed in the LacZ assay described below with the PgIpF promoter run as positive reference in the same assay. To compare across assays the activity is calculated relative to the PgIpF promoter, a range indicates results from multiple assays.

The promoter may be of heterologous origin, native to the genetically modified cell or it may be a recombinant promoter, combining heterologous and/or native elements.

One way to increase the production of a product may be to regulate the production of the desired enzyme activity used to produce the product, such as the glycosyltransferases or enzymes involved in the biosynthetic pathway of the glycosyl donor.

Increasing the promoter strength driving the expression of the desired enzyme may be one way of doing this. The strength of a promoter can be assed using a lacZ enzyme assay where β-galactosidase activity is assayed as described previously (see e.g. Miller J. H. Experiments in molecular genetics, Cold spring Harbor Laboratory Press, NY, 1972). Briefly the cells are diluted in Z-buffer and permeabilized with sodium dodecyl sulfate (0.1%) and chloroform. The LacZ assay is performed at 30° C. Samples are preheated, the assay initiated by addition of 200 μl ortho-nitro-phenyl-β-galactosidase (4 mg/ml) and stopped by addition of 500 μl of 1 M Na₂CO₃ when the sample had turned slightly yellow. The release of ortho-nitrophenol is subsequently determined as the change in optical density at 420 nm. The specific activities are reported in Miller Units (MU) [A420/(min*ml*A600)]. A regulatory element with an activity above 10,000 MU is considered strong and a regulatory element with an activity below 3,000 MU is considered weak, what is in between has intermediate strength. An example of a strong regulatory element is the PglpF promoter with an activity of approximately 14.000 MU and an example of a weak promoter is Plac which when induced with IPTG has an activity of approximately 2300 MU.

In embodiments the expression of said nucleic acid sequences of the present disclosure is under control of a PglpF (SEQ ID NO: 15) or Plac (SEQ ID NO: 19) promoter or PmglB_UTR70 (SEQ ID NO: 21) or PglpA_70UTR (SEQ ID NO: 47) or PglpT_70UTR (SEQ ID NO: 48) or variants thereof such as promoters identified in Table 2, in particular PglpF variants of SEQ ID NO: 45, 49, 50, 51, 53, 18 or 54 or Plac variant of SEQ ID NO: 20 or PmglB_70UTR variants of SEQ ID NO: 21, 22, 23, 41, 42, 43, 44, 46 or 52. Further suitable variants of PglpF, PglpA_70UTR, PglpT_70UTR and PmglB_70UTR promoter sequences are described in or WO2019/123324 and WO2020/255054 respectively (hereby incorporated by reference).

Integration of the nucleic acid construct of interest comprised in the construct (expression cassette) into the bacterial genome can be achieved by conventional methods, e.g. by using linear cartridges that contain flanking sequences homologous to a specific site on the chromosome, as described for the attTn7-site (Waddell C. S. and Craig N. L., Genes Dev. (1988) February; 2(2):137-49.); methods for genomic integration of nucleic acid sequences in which recombination is mediated by the Red recombinase function of the phage λ or the RecE/RecT recombinase function of the Rac prophage (Murphy, J Bacteriol. (1998); 180(8):2063-7; Zhang et al., Nature Genetics (1998) 20: 123-128 Muyrers et al., EMBO Rep. (2000) 1(3): 239-243); methods based on Red/ET recombination (Wenzel et al., Chem Biol. (2005), 12(3):349-56.; Vetcher et al., Appl Environ Microbiol. (2005); 71(4):1829-35); or positive clones, i.e., clones that carry the expression cassette, can be selected e.g., by means of a marker gene, or loss or gain of gene function.

In one or more exemplary embodiments, the present disclosure relates to one or more recombinant nucleic acid sequences as illustrated in SEQ ID NOs: 24, 25, 26, 27 or 28 [nucleic acid encoding Ccol2, Cjej1, Csub1, Chepa, and Clari1, respectively].

In particular, the present disclosure relates to one or more of a recombinant nucleic acid sequence and/or to a functional homologue thereof having a sequence which is at least 70% identical to SEQ ID NOs: 24, 25, 26, 27 or 28 [nucleic acids encoding Ccol2, Cjej1, Csub1, Chepa, and Clari1, respectively], such as at least 75% identical, at least 80% identical, at least 85% identical, at least 90% identical, at least, at least 95% identical, at least 98% identical, or 100% identical.

### Sequence Identity

The term “sequence identity” as used herein describes the relatedness between two amino acid sequences or between two nucleotide sequences, i.e., a candidate sequence (e.g., a sequence described herein) and a reference sequence (such as a prior art sequence) based on their pairwise alignment. For purposes of the present disclosure, the sequence identity between two amino acid sequences is determined using the Needleman-Wunsch algorithm (Needleman and Wunsch, 1970, J. Mo/. Biol. 48: 443-453) as implemented in the Needle program of the EMBOSS package (EMBOSS: The European Molecular Biology Open Software Suite, Rice et al., 2000, Trends Genet. 16: 276-277,), preferably version 5.0.0 or later (available at https://www.ebi.ac.uk/Tools/psa/emboss needle/). The parameters used are gap open penalty of 10, gap extension penalty of 0.5, and the EBLOSUM62 (EMBOSS version of 30 BLOSUM62) substitution matrix. The output of Needle labeled “longest identity” (obtained using the -nobrief option) is used as the percent identity and is calculated as follows: (Identical Residues×100)/(Length of Alignment−Total Number of Gaps in Alignment).

For purposes of the present disclosure, the sequence identity between two nucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, Trends Genet. 16: 276-277), 10 preferably version 5.0.0 or later. The parameters used are gap open penalty of 10, gap extension penalty of 0.5, and the DNAFULL (EMBOSS version of NCBI NUC4.4) substitution matrix. The output of Needle labeled “longest identity” (obtained using the -nobrief option) is used as the percent identity and is calculated as follows: (Identical Deoxyribonucleotides×100)/(Length of Alignment−Total Number of Gaps in Alignment).

### Functional Homologue

A functional homologue or functional variant of a protein/nucleic acid sequence as described herein is a protein/nucleic acid sequence with alterations in the genetic code, which retain its original functionality. A functional homologue may be obtained by mutagenesis or may be natural occurring variants from the same or other species. The functional homologue should have a remaining functionality of at least 50%, such as at least 60%, 70%, 80%, 90% or 100% compared to the functionality of the protein/nucleic acid sequence.

A functional homologue of any one of the disclosed amino acid or nucleic acid sequences can also have a higher functionality. A functional homologue of any one of the amino acid sequences shown in table 1 or a recombinant nucleic acid encoding any one of the sequences of table 4, should ideally be able to participate in the production of sialylated HMOs, in terms of increased HMO yield, export of HMO product out of the cell or import of substrate for the HMO production, such as a acceptor oligosaccharide of at least three monosaccharide units, improved purity/by-product formation, reduction in biomass formation, viability of the genetically engineered cell, robustness of the genetically engineered cell according to the disclosure, or reduction in consumables needed for the production.

### Use of a Genetically Modified Cell

The disclosure also relates to any commercial use of the genetically modified cell(s) or the nucleic acid construct(s) disclosed herein, such as, but not limited to, in a method for producing a sialylated human milk oligosaccharide (HMO).

In an exemplified embodiment, the genetically modified cell and/or the nucleic acid construct according to the disclosure is used in the manufacturing of HMOs. Preferably, in the manufacturing of HMOs, wherein the molar % content of LST-a produced by the genetically modified cell is above 9% of the total HMO.

In an exemplified embodiment, the genetically modified cell and/or the nucleic acid construct according to the disclosure is used in the manufacturing of one or more sialylated HMO(s), wherein the sialylated HMOs are 3′SL and/or LST-a.

In an exemplified embodiment, the genetically modified cell and/or the nucleic acid construct according to the disclosure is used in the manufacturing of a mixture of HMO(s), comprising at least two HMOs selected from 3′SL, LNT-II, LNT and LST-a.

In an exemplified embodiment, the genetically modified cell and/or the nucleic acid construct according to the disclosure is used in the manufacturing of a mixture of HMO(s), comprising or consisting of 3′SL, LNT-II, LNT and/or LST-a.

In an exemplified embodiment, the genetically modified cell and/or the nucleic acid construct according to the disclosure is used in the manufacturing of a mixture of HMO(s), comprising 3′SL and LST-a.

In an exemplified embodiment, the genetically modified cell and/or the nucleic acid construct according to the disclosure is used in the manufacturing of one or more sialylated HMO(s), wherein the HMOs are 3′SL and/or LST-a.

In one or more embodiments, the genetically engineered cell and/or the nucleic acid construct is used in the manufacturing of 3′SL.

In one or more exemplary embodiments, the genetically engineered cell and/or the nucleic acid construct is used in the manufacturing of LST-a.

Production of these HMO's may require the presence of two or more glycosyltransferase activities.

### A Method for Producing Sialylated Human Milk Oligosaccharides (HMOs)

The present disclosure also relates to a method for producing a sialylated human milk oligosaccharide (HMO), said method comprises culturing a genetically modified cell according to the present disclosure.

The present disclosure relates to a method for producing human milk oligosaccharides (HMOs), wherein the molar % content of LST-a produced by the genetically modified cell is above 9% of the total HMO.

The present disclosure relates to a method for producing human milk oligosaccharides (HMOs), wherein the molar % content of LST-a produced by the genetically modified cell is above 9% of the total HMO and the molar % content of 3′SL produced by the genetically modified cell is below 20%, such as below 10%.

The present disclosure thus relates to a method for producing a sialylated human milk oligosaccharide (HMO), said method comprising culturing a genetically modified cell, said cell comprising:
a recombinant nucleic acid sequence encoding an enzyme with α-2,3-sialyltransferase activity, wherein said enzyme is selected from the group consisting of:
a. Ccol2 comprising or consisting of the amino acid sequence of SEQ ID NO: 1 or an amino acid sequence with at least 80%, such as at least 85%, such as at least 90%, such as at least 95%, or such as at least 99% identity to SEQ ID NO: 1,
b. Cjej1 comprising or consisting of the amino acid sequence of SEQ ID NO: 2 or an amino acid sequence with at least 80%, such as at least 85%, such as at least 90%, such as at least 95%, or such as at least 99% identity to SEQ ID NO: 2,
c. Csub1 comprising or consisting of the amino acid sequence of SEQ ID NO: 3 or an amino acid sequence with at least 80%, such as at least 85%, such as at least 90%, such as at least 95%, or such as at least 99% identity to SEQ ID NO: 3,
d. Chepa comprising or consisting of the amino acid sequence of SEQ ID NO: 4 or an amino acid sequence with at least 80%, such as at least 85%, such as at least 90%, such as at least 95%, or such as at least 99% identity to SEQ ID NO: 4, and/or
e. Clari1 comprising or consisting of the amino acid sequence of SEQ ID NO: 5 or an amino acid sequence with at least 80%, such as at least 85%, such as at least 90%, such as at least 95%, or such as at least 99% identity to SEQ ID NO: 5; and
wherein said cell produces a sialylated HMO.

In one or more exemplary embodiments, the α-2,3-sialyltransferase of the present disclosure is under control of a PglpF, a Plac, or a PmglB_70UTR, a PglpA_70UTR, or a PglpT_70UTR promoter. Thus, in an exemplary embodiment, the α-2,3-sialyltransferase of the present disclosure is under control of a PglpF promoter or a variant thereof (table 2). In another exemplary embodiment, the α-2,3-sialyltransferase of the present disclosure is under control of a PmglB promoter or a variant thereof (table 2). Preferably, the recombinant nucleic acid encoding an enzyme with α-2,3-sialyltransferase is under control of a strong promoter selected from the group consisting of SEQ ID NOs 15, 20, 21, 22, 23, 41, 42, 43, 44, 45, 46, 47, 48, 49, 50, 51, and 52.

Further genetic modifications can e.g., be selected from inclusion of additional glycosyltransferases and/or metabolic pathway engineering, and inclusion of MFS transporters, as described in the above sections, which the skilled person will know how to combine into a genetically modified cell capable of producing one or more sialylated HMO's.

The method particularly comprises culturing a genetically modified cell that produces a sialylated HMO, wherein the LST-a content produced by said cell is at least 9% of the total HMO content produced by the cell. In addition, the method comprises culturing a genetically modified cell that produces a sialylated HMO, wherein the 3′SL content produced by said cell is below 30%, such as below 25%, such as below 20%, such as below 15%, such as below 12%, such as below 11%, such as below 10%, such as between 8% to 12% of the total HMO content produced by the cell.

The method comprising culturing a genetically modified cell that produces a sialylated HMO and further comprises culturing said genetically engineered cell in in the presence of an energy source (carbon source) selected from the group consisting of glucose, sucrose, fructose, xylose and glycerol.

In one aspect, the method according to the present disclosure produces a sialylated human milk oligosaccharide (HMO), such as 3′SL and/or LST-a.

In one aspect, the method according to the present disclosure produces, one or more HMO(s), wherein the HMOs are 3′SL, LNT and/or LST-a.

In one aspect, the method according to the present disclosure, produces a mixture of HMO(s), comprising at least two HMOs, such as at least three HMOs selected from 3′SL, LNT-II, LNT and LST-a.

In one aspect, the method according to the present disclosure produces a mixture of HMO(s), comprising at least two HMOs selected from 3′SL, LNT-II, LNT and LST-a.

In one aspect, the method according to the present disclosure produces a mixture of HMO(s), comprising or consisting of 3′SL, LNT-II, LNT, and LST-a.

In one aspect, the method according to the present disclosure produces a mixture of HMO(s), comprising 3′SL and LST-a.

In one aspect, the method according to the present disclosure produces one or more sialylated HMO(s), wherein the HMOs are 3′SL, LST-a and/or DS-LNT.

In one aspect, the method according to the present disclosure produces one or more sialylated HMO(s), wherein the HMOs are 3′SL and/or LST-a.

In one aspect, the method according to the present disclosure produces 3′SL.

In one aspect, the method according to the present disclosure produces LST-a.

To enable the production of sialylated HMOs in the method according to the present disclosure, the genetically modified cell may comprise a biosynthetic pathway for making a sialic acid sugar nucleotide, alternatively sialic acid can be added during cultivation of the cell.

In preferred embodiments of the methods of the present disclosure, the genetically modified cell comprises a biosynthetic pathway for making a sialic acid sugar nucleotide. Preferably, in methods of the present disclosure, the sialic acid sugar nucleotide is CMP-Neu5Ac. Thus, in methods of the present disclosure the sugar nucleotide pathway is expressed by the genetically modified cell, wherein the CMP-Neu5Ac pathway is encoded by the neuBCA operon from Campylobacter jejuni of SEQ ID NO: 38. In methods of the present disclosure, the sialic acid sugar nucleotide pathway is encoded from a high-copy plasmid bearing the neuBCA operon.

The method of the present disclosure comprises providing a glycosyl donor, which is synthesized separately by one or more genetically engineered cells and/or is exogenously added to the culture medium from an alternative source.

In one aspect, the method of the present disclosure further comprises providing an acceptor saccharide as substrate for the HMO formation, the acceptor saccharide comprising at least two monosaccharide units, which is exogenously added to the culture medium and/or has been produced by a separate microbial fermentation.

In one aspect, the method of the present disclosure comprises providing an acceptor saccharide comprising at least two monosaccharide units, which is exogenously added to the culture medium and/or has been produced by a separate microbial fermentation and which is selected form lactose, LNT-II and LNT. In a preferred embodiment the substrate for HMO formation is lactose which is fed to the culture during the fermentation of the genetically engineered cell.

The sialylated human milk oligosaccharide (HMO) is retrieved from the culture, either from the culture medium and/or the genetically modified cell.

In particular, the present disclosure relates to a method for producing LST-a, said method comprising:
a) obtaining a genetically modified cell comprising
   i. a recombinant nucleic acid sequence, preferably under control of a PglpF promoter, encoding an enzyme with α-2,3-sialyltransferase activity, wherein said enzyme is selected from the group consisting of: Ccol2 comprising or consisting of the amino acid sequence of SEQ ID NO: 1 or an amino acid sequence with at least 80%, such as at least 85%, such as at least 90%, such as at least 95%, or such as at least 99% identity to SEQ ID NO: 1, Cjej1 comprising or consisting of the amino acid sequence of SEQ ID NO: 2 or an amino acid sequence with at least 80%, such as at least 85%, such as at least 90%, such as at least 95%, or such as at least 99% identity to SEQ ID NO: 2, Csub1 comprising or consisting of the amino acid sequence of SEQ ID NO: 3 or an amino acid sequence with at least 80%, such as at least 85%, such as at least 90%, such as at least 95%, or such as at least 99% identity to SEQ ID NO: 3, Chepa comprising or consisting of the amino acid sequence of SEQ ID NO: 4 or an amino acid sequence with at least 80%, such as at least 85%, such as at least 90%, such as at least 95%, or such as at least 99% identity to SEQ ID NO: 4, and Clari1 comprising or consisting of the amino acid sequence of SEQ ID NO: 5 or an amino acid sequence with at least 80%, such as at least 85%, such as at least 90%, such as at least 95%, or such as at least 99% identity to SEQ ID NO: 5; and
   ii. at least one nucleic acid sequence encoding a heterologous β-1,3-galactosyltransferase, such as GaITK from Helicobacter pylori, preferably under control of a PglpF promoter,
   iii. optionally, a nuclei acid sequence encoding a β-1,3-N-acetyl-glucosaminyl-transferase, such as LgtA from Neisseria meningitidis, preferably under control of a PglpF promoter and
   iv. optionally, a nucleic acid sequence encoding an MFS transporter, such as but not limited to Fred, Nec and/or yberC, preferably under control of a PglpF or Plac promoter, and
b) culturing said genetically modified cell in a carbon-source containing culture medium and in the presence of lactose or LNT-II, and
c) producing said sialylated human milk oligosaccharide (HMO), in particular LST-a, by said genetically modified cell, and
d) retrieving the sialylated human milk oligosaccharide (HMO), in particular LST-a, from the culture medium and/or the genetically modified cell.

In particular, the present disclosure relates to a method for producing LST-a, said method comprising:
a) obtaining a genetically modified cell comprising
   i. a recombinant nucleic acid sequence encoding an enzyme with α-2,3-sialyltransferase activity, wherein said enzyme is Ccol2 comprising or consisting of the amino acid sequence of SEQ ID NO: 1 or an amino acid sequence with at least 80%, such as at least 85%, such as at least 90%, such as at least 95%, or such as at least 99% identity to SEQ ID NO: 1 and
   ii. at least one nucleic acid sequence encoding a heterologous β-1,3-galactosyltransferase that is GaITK from Helicobacter pylori, under control of a PglpF promoter,
   iii. at least one a nucleic acid sequence encoding a β-1,3-N-acetyl-glucosaminyl-transferase, that is LgtA from Neisseria meningitidis, under control of a PglpF promoter, and
b) culturing said genetically modified cell in a carbon-source containing culture medium and in the presence of lactose or LNT-II, and
c) producing said sialylated human milk oligosaccharide (HMO), in particular LST-a, by said genetically modified cell, and
d) retrieving the sialylated human milk oligosaccharide (HMO), in particular LST-a, from the culture medium and/or the genetically modified cell.

In particular, the present disclosure relates to a method for producing LST-a, said method comprising:
a) obtaining a genetically modified cell comprising
   i. a recombinant nucleic acid sequence encoding an enzyme with α-2,3-sialyltransferase activity, wherein said enzyme is Cjej1 comprising or consisting of the amino acid sequence of SEQ ID NO: 2 or an amino acid sequence with at least 80%, such as at least 85%, such as at least 90%, such as at least 95%, or such as at least 99% identity to SEQ ID NO: 2; and
   ii. at least one nucleic acid sequence encoding a heterologous β-1,3-galactosyltransferase that is GaITK from Helicobacter pylori, under control of a PglpF promoter,
   iii. at least one a nucleic acid sequence encoding a β-1,3-N-acetyl-glucosaminyl-transferase, that is LgtA from Neisseria meningitidis, under control of a PglpF promoter, and
b) culturing said genetically modified cell in a carbon-source containing culture medium and in the presence of lactose or LNT-II, and
c) producing said sialylated human milk oligosaccharide (HMO), in particular LST-a, by said genetically modified cell, and
d) retrieving the sialylated human milk oligosaccharide (HMO), in particular LST-a, from the culture medium and/or the genetically modified cell.

In particular, the present disclosure relates to a method for producing LST-a, said method comprising:
a) obtaining a genetically modified cell comprising
   i. a recombinant nucleic acid sequence encoding an enzyme with α-2,3-sialyltransferase activity, wherein said enzyme is Csub1 comprising or consisting of the amino acid sequence of SEQ ID NO: 3 or an amino acid sequence with at least 80%, such as at least 85%, such as at least 90%, such as at least 95%, or such as at least 99% identity to SEQ ID NO: 3 and
   ii. at least one nucleic acid sequence encoding a heterologous β-1,3-galactosyltransferase that is GaITK from Helicobacter pylori, under control of a PglpF promoter,
   iii. at least one a nucleic acid sequence encoding a β-1,3-N-acetyl-glucosaminyl-transferase, that is LgtA from Neisseria meningitidis, under control of a PglpF promoter, and
b) culturing said genetically modified cell in a carbon-source containing culture medium and in the presence of lactose or LNT-II, and
c) producing said sialylated human milk oligosaccharide (HMO), in particular LST-a, by said genetically modified cell, and
d) retrieving the sialylated human milk oligosaccharide (HMO), in particular LST-a, from the culture medium and/or the genetically modified cell.

In particular, the present disclosure relates to a method for producing LST-a, said method comprising:
a) obtaining a genetically modified cell comprising
   i. a recombinant nucleic acid sequence encoding an enzyme with α-2,3-sialyltransferase activity, wherein said enzyme is Chepa comprising or consisting of the amino acid sequence of SEQ ID NO: 4 or an amino acid sequence with at least 80%, such as at least 85%, such as at least 90%, such as at least 95%, or such as at least 99% identity to SEQ ID NO: 4, and
   ii. at least one nucleic acid sequence encoding a heterologous β-1,3-galactosyltransferase that is GaITK from Helicobacter pylori, under control of a PglpF promoter,
   iii. at least one a nucleic acid sequence encoding a β-1,3-N-acetyl-glucosaminyl-transferase, that is LgtA from Neisseria meningitidis, under control of a PglpF promoter, and
b) culturing said genetically modified cell in a carbon-source containing culture medium and in the presence of lactose or LNT-II, and
c) producing said sialylated human milk oligosaccharide (HMO), in particular LST-a, by said genetically modified cell, and
d) retrieving the sialylated human milk oligosaccharide (HMO), in particular LST-a, from the culture medium and/or the genetically modified cell.

In particular, the present disclosure relates to a method for producing LST-a, said method comprising:
a) obtaining a genetically modified cell comprising
   i. a recombinant nucleic acid sequence encoding an enzyme with α-2,3-sialyltransferase activity, wherein said enzyme is Clari1 comprising or consisting of the amino acid sequence of SEQ ID NO: 5 or an amino acid sequence with at least 80%, such as at least 85%, such as at least 90%, such as at least 95%, or such as at least 99% identity to SEQ ID NO: 5; and
   ii. at least one nucleic acid sequence encoding a heterologous β-1,3-galactosyltransferase that is GaITK from Helicobacter pylori, under control of a PglpF promoter,
   iii. at least one a nucleic acid sequence encoding a β-1,3-N-acetyl-glucosaminyl-transferase, that is LgtA from Neisseria meningitidis, under control of a PglpF promoter, and
b) culturing said genetically modified cell in a carbon-source containing culture medium and in the presence of lactose or LNT-II, and
c) producing said sialylated human milk oligosaccharide (HMO), in particular LST-a, by said genetically modified cell, and
d) retrieving the sialylated human milk oligosaccharide (HMO), in particular LST-a, from the culture medium and/or the genetically modified cell.

In particular, the present disclosure relates to a method for producing 3′SL and LST-a, said method comprising:
a) obtaining a genetically modified cell comprising
   i. a recombinant nucleic acid sequence encoding an enzyme with α-2,3-sialyltransferase activity, wherein said enzyme is selected from the group consisting of: Ccol2 comprising or consisting of the amino acid sequence of SEQ ID NO: 1 or an amino acid sequence with at least 80%, such as at least 85%, such as at least 90%, such as at least 95%, or such as at least 99% identity to SEQ ID NO: 1, Cjej1 comprising or consisting of the amino acid sequence of SEQ ID NO: 2 or an amino acid sequence with at least 80%, such as at least 85%, such as at least 90%, such as at least 95%, or such as at least 99% identity to SEQ ID NO: 2, Csub1 comprising or consisting of the amino acid sequence of SEQ ID NO: 3 or an amino acid sequence with at least 80%, such as at least 85%, such as at least 90%, such as at least 95%, or such as at least 99% identity to SEQ ID NO: 3, Chepa comprising or consisting of the amino acid sequence of SEQ ID NO: 4 or an amino acid sequence with at least at least 80%, such as at least 85%, such as at least 90%, such as at least 95%, or such as at least 99% identity to SEQ ID NO: 4, and Clari1 comprising or consisting of the amino acid sequence of SEQ ID NO: 5 or an amino acid sequence with at least 80%, such as at least 85%, such as at least 90%, such as at least 95%, or such as at least 99% identity to SEQ ID NO: 5; and
   ii. at least one nucleic acid sequence encoding a heterologous β-1,3-galactosyltransferase, such as GaITK from Helicobacter pylori, preferably under control of a PglpF promoter,
   iii. optionally, a nucleic acid sequence encoding a β-1,3-N-acetyl-glucosaminyl-transferase, such as LgtA from Neisseria meningitidis, preferably under control of a PglpF promoter and
   iv. optionally, a nucleic acid sequence encoding an MFS transporter such as but not limited to Fred, Nec and/or yberC, preferably under control of a PglpF or Plac promoter, and
b) culturing said genetically modified cell in a carbon-source containing culture medium and in the presence of lactose, and
c) producing said sialylated human milk oligosaccharides (HMO) 3′SL and LST-a, by said genetically modified cell, and
d) retrieving the sialylated human milk oligosaccharides (HMO) 3′SL and LST-a, from the culture medium and/or the genetically modified cell.

In particular, the present disclosure relates to a method for producing 3′SL and LST-a, said method comprising:
a) obtaining a genetically modified cell comprising
   i. a recombinant nucleic acid sequence encoding an enzyme with α-2,3-sialyltransferase activity, wherein said enzyme is Ccol2 comprising or consisting of the amino acid sequence of SEQ ID NO: 1 or an amino acid sequence with at least 80%, such as at least 85%, such as at least 90%, such as at least 95%, or such as at least 99% identity to SEQ ID NO: 1 and
   ii. at least one nucleic acid sequence encoding a heterologous β-1,3-galactosyltransferase that is GaITK from Helicobacter pylori, under control of a PglpF promoter,
   iii. at least one a nucleic acid sequence encoding a β-1,3-N-acetyl-glucosaminyl-transferase, that is LgtA from Neisseria meningitidis, under control of a PglpF promoter, and
b) culturing said genetically modified cell in a carbon-source containing culture medium and in the presence of lactose, and
c) producing said sialylated human milk oligosaccharides (HMO) 3′SL and LST-a, by said genetically modified cell, and
d) retrieving the sialylated human milk oligosaccharides (HMO) 3′SL and LST-a, from the culture medium and/or the genetically modified cell.

In particular, the present disclosure relates to a method for producing 3′SL and LST-a, said method comprising:
a) obtaining a genetically modified cell comprising
   i. a recombinant nucleic acid sequence encoding an enzyme with α-2,3-sialyltransferase activity, wherein said enzyme is Cjej1 comprising or consisting of the amino acid sequence of SEQ ID NO: 2 or an amino acid sequence with at least 80%, such as at least 85%, such as at least 90%, such as at least 95%, or such as at least 99% identity to SEQ ID NO: 2; and
   ii. at least one nucleic acid sequence encoding a heterologous β-1,3-galactosyltransferase that is GaITK from Helicobacter pylori, under control of a PglpF promoter,
   iii. at least one a nucleic acid sequence encoding a β-1,3-N-acetyl-glucosaminyl-transferase, that is LgtA from Neisseria meningitidis, under control of a PglpF promoter, and
b) culturing said genetically modified cell in a carbon-source containing culture medium and in the presence of lactose, and
c) producing said sialylated human milk oligosaccharides (HMO) 3′SL and LST-a, by said genetically modified cell, and
d) retrieving the sialylated human milk oligosaccharides (HMO) 3′SL and LST-a from the culture medium and/or the genetically modified cell.

In particular, the present disclosure relates to a method for producing 3′SL and LST-a, said method comprising:
a) obtaining a genetically modified cell comprising
   i. a recombinant nucleic acid sequence encoding an enzyme with α-2,3-sialyltransferase activity, wherein said enzyme is Csub1 comprising or consisting of the amino acid sequence of SEQ ID NO: 3 or an amino acid sequence with at least 80%, such as at least 85%, such as at least 90%, such as at least 95%, or such as at least 99% identity to SEQ ID NO: 3 and
   ii. at least one nucleic acid sequence encoding a heterologous β-1,3-galactosyltransferase that is GaITK from Helicobacter pylori, under control of a PglpF promoter,
   iii. at least one a nucleic acid sequence encoding a β-1,3-N-acetyl-glucosaminyl-transferase, that is LgtA from Neisseria meningitidis, under control of a PglpF promoter, and
b) culturing said genetically modified cell in a carbon-source containing culture medium and in the presence of lactose, and
c) producing said sialylated human milk oligosaccharides (HMOs) 3′SL and LST-a, by said genetically modified cell, and
d) retrieving the sialylated human milk oligosaccharides (HMOs) 3′SL and LST-a, from the culture medium and/or the genetically modified cell.

In particular, the present disclosure relates to a method for producing 3′SL and LST-a, said method comprising:
a) obtaining a genetically modified cell comprising
   i. a recombinant nucleic acid sequence encoding an enzyme with α-2,3-sialyltransferase activity, wherein said enzyme is Chepa comprising or consisting of the amino acid sequence of SEQ ID NO: 4 or an amino acid sequence with at least 80%, such as at least 85%, such as at least 90%, such as at least 95%, or such as at least 99% identity to SEQ ID NO: 4, and
   ii. at least one nucleic acid sequence encoding a heterologous β-1,3-galactosyltransferase that is GaITK from Helicobacter pylori, under control of a PglpF promoter,
   iii. at least one a nucleic acid sequence encoding a β-1,3-N-acetyl-glucosaminyl-transferase, that is LgtA from Neisseria meningitidis, under control of a PglpF promoter, and
b) culturing said genetically modified cell in a carbon-source containing culture medium and in the presence of lactose, and
c) producing said sialylated human milk oligosaccharides (HMOs) 3′SL and LST-a, by said genetically modified cell, and
d) retrieving the sialylated human milk oligosaccharides (HMOs) 3′SL and LST-a, from the culture medium and/or the genetically modified cell.

In particular, the present disclosure relates to a method for producing 3′SL and LST-a, said method comprising:
a) obtaining a genetically modified cell comprising
   i. a recombinant nucleic acid sequence encoding an enzyme with α-2,3-sialyltransferase activity, wherein said enzyme is Clari1 comprising or consisting of the amino acid sequence of SEQ ID NO: 5 or an amino acid sequence with at least 80%, such as at least 85%, such as at least 90%, such as at least 95%, or such as at least 99% identity to SEQ ID NO: 5; and
   ii. at least one nucleic acid sequence encoding a heterologous β-1,3-galactosyltransferase that is GaITK from Helicobacter pylori, under control of a PglpF promoter,
   iii. at least one a nucleic acid sequence encoding a β-1,3-N-acetyl-glucosaminyl-transferase, that is LgtA from Neisseria meningitidis, under control of a PglpF promoter, and
b) culturing said genetically modified cell in a carbon-source containing culture medium and in the presence of lactose, and
c) producing said sialylated human milk oligosaccharides (HMOs) 3′SL and LST-a, by said genetically modified cell, and
d) retrieving the sialylated human milk oligosaccharides (HMOs), 3′SL and LST-a, from the culture medium and/or the genetically modified cell.

Culturing or fermenting (used interchangeably herein) in a controlled bioreactor typically comprises (a) a first phase of exponential cell growth in a culture medium ensured by a carbon-source, and (b) a second phase of cell growth in a culture medium run under carbon limitation, where the carbon-source is added continuously together with the acceptor oligosaccharide, such as lactose, allowing formation of the HMO product in this phase. By carbon (sugar) limitation is meant the stage in the fermentation where the growth rate is kinetically controlled by the concentration of the carbon source (sugar) in the culture broth, which in turn is determined by the rate of carbon addition (sugar feed-rate) to the fermenter.

The terms “manufacturing” or “manufacturing scale” or “large-scale production” or “large-scale fermentation”, are used interchangeably and defines a fermentation with a minimum volume of 100 L, such as 1000 L, such as 10.000 L, such as 100.000 L, such as 200.000 L culture broth. Usually, a “manufacturing scale” process is defined by being capable of processing large volumes yielding amounts of the HMO product of interest that meet, e.g., in the case of a therapeutic compound or composition, the demands for toxicity tests, clinical trials as well as for market supply. In addition to the large volume, a manufacturing scale method, as opposed to simple lab scale methods like shake flask cultivation, is characterized by the use of the technical system of a bioreactor (fermenter) which is equipped with devices for agitation, aeration, nutrient feeding, monitoring and control of process parameters (pH, temperature, dissolved oxygen tension, back pressure, etc.). To a large extent, the behavior of an expression system in a lab scale method, such as shake flasks, benchtop bioreactors or the deep well format described in the examples of the disclosure, does allow to predict the behavior of that system in the complex environment of a bioreactor.

With regards to the suitable cell medium used in the fermentation process, there are no limitations. The culture medium may be semi-defined, i.e., containing complex media compounds (e.g., yeast extract, soy peptone, casamino acids, etc.), or it may be chemically defined, without any complex compounds. The carbon-source can be selected from the group consisting of glucose, sucrose, fructose, xylose and glycerol. In one or more exemplary embodiments, the culturing media is supplemented with one or more energy and carbon sources selected form the group containing glycerol, sucrose and glucose.

In one or more exemplary embodiments, the culturing media contains sucrose as the sole carbon and energy source. In one or more exemplary embodiments, the genetically engineered cell comprises one or more heterologous nucleic acid sequence encoding one or more heterologous polypeptide(s) which enables utilization of sucrose as sole carbon and energy source of said genetically engineered cell.

In one or more exemplary embodiments, the genetically engineered cell comprises a PTS-dependent sucrose utilization system, further comprising the scrYA and scrBR operons as described in WO2015/197082 (hereby incorporated by reference).

After carrying out the method of this disclosure, the sialylated HMO produced can be collected from the cell culture or fermentation broth in a conventional manner.

### Retrieving/Harvesting

The sialylated human milk oligosaccharide (HMO) is retrieved from the culture medium and/or the genetically modified cell. In the present context, the term “retrieving” is used interchangeably with the term “harvesting”. Both “retrieving” and “harvesting” in the context relate to collecting the produced HMO(s) from the culture/broth following the termination of fermentation. In one or more exemplary embodiments it may include collecting the HMO(s) included in both the biomass (i.e., the host cells) and cultivation media, i.e., before/without separation of the fermentation broth from the biomass. In other embodiments, the produced HMOs may be collected separately from the biomass and fermentation broth, i.e., after/following the separation of biomass from cultivation media (i.e., fermentation broth).

The separation of cells from the medium can be carried out with any of the methods well known to the skilled person in the art, such as any suitable type of centrifugation or filtration. The separation of cells from the medium can follow immediately after harvesting the fermentation broth or be carried out at a later stage after storing the fermentation broth at appropriate conditions. Recovery of the produced HMO(s) from the remaining biomass (or total fermentation broth) include extraction thereof from the biomass (i.e., the production cells).

After recovery from fermentation, HMO(s) are available for further processing and purification.

The HMOs can be purified according to the procedures known in the art, e.g., such as described in WO2017/182965 or WO2017/152918, wherein the latter describes purification of sialylated HMOs. The purified HMOs can be used as nutraceuticals, pharmaceuticals, or for any other purpose, e.g., for research.

In embodiments LST-a is further purified from the recovery from the fermentation to produce at least 75%, such as at least 80%, such as at least 85%, such as at least 90%, such as at least 95% pure LST-a.

At the end of culturing, the oligosaccharide as product can be accumulated both in the intra- and the extracellular matrix.

The method according to the present disclosure comprises cultivating the genetically engineered microbial cell in a culture medium which is designed to support the growth of microorganisms, and which contains one or more carbohydrate sources or just carbon-source, such as selected from the group consisting of glucose, sucrose, fructose, xylose and glycerol.

In one or more exemplary embodiments, the culturing media is supplemented with one or more energy and carbon sources selected form the group containing glycerol, sucrose and glucose.

### Manufactured Product

The term “manufactured product” according to the use of the genetically engineered cell or the nucleic acid construct refer to the one or more HMOs intended as the one or more product HMO(s). The various products are described above.

Advantageously, the methods disclosed herein provide both a decreased ratio of by-product to product and an increased overall yield of the product (and/or HMOs in total). This, less by-product formation in relation to product formation, facilitates an elevated product production and increases efficiency of both the production and product recovery process, providing superior manufacturing procedure of HMOs.

The manufactured product may be a powder, a composition, a suspension, or a gel comprising one or more HMOs.

### Sequences

The current application contains a sequence listing in text format and electronical format which is hereby incorporated by reference.

An overview of the SEQ ID NOs used in the present application can be found in table 1 (alpha-2,3-sialyltransferase protein sequences), 2 (promoter sequences) and 4 (alpha-2,3-sialyltransferase DNA sequences), additional sequences described in the application is the DNA sequence encoding the neuBCA operon from Campylobacter jejuni (SEQ ID NO: 38) and the @-1,3-N-acetylglucosaminyltransferase LgtA from N. meningitidis (SEQ ID NO: 39) and the β-1,3-galactosyltransferases galTK from H. pylori (SEQ ID NO: 40) and the lacY sequence from E. coli (SEQ ID NO: 56).

### Embodiments

Various embodiments of present disclosure are described in the following items.
1. A genetically modified cell comprising a recombinant nucleic acid sequence encoding an enzyme with α-2,3-sialyltransferase activity, which is capable of producing at least 9% LST-a of the total molar HMO content produced by the cell.
2. The genetically modified cell according to item 1, wherein said α-2,3-sialyltransferase enzyme is selected from the group consisting of:
   a. Ccol2 comprising or consisting of the amino acid sequence of SEQ ID NO: 1 or an amino acid sequence with at least 80% identity to SEQ ID NO: 1,
   b. Cjej1 comprising or consisting of the amino acid sequence of SEQ ID NO: 2 or an amino acid sequence with at least 80% identity to SEQ ID NO: 2,
   c. Csub1 comprising or consisting of the amino acid sequence of SEQ ID NO: 3 or an amino acid sequence with at least 80% identity to SEQ ID NO: 3,
   d. Chepa comprising or consisting of the amino acid sequence of SEQ ID NO: 4 or an amino acid sequence with at least 80% identity to SEQ ID NO: 4, and
   e. Clari1 comprising or consisting of the amino acid sequence of SEQ ID NO: 5 or an amino acid sequence with at least 80% identity to SEQ ID NO: 5.
3. The genetically modified cell according to any one of items 1 or 2, wherein the cell produces LST-a and 3′SL when lactose is used as initial substrate for the HMO formation.
4. The genetically modified cell according to any of the preceding items, wherein the 3′SL produced does not exceed 20% of the total molar content of the HMOs produced by said cell.
5. The genetically modified cell according to any of the preceding items, wherein the sialyltransferase is under the control of a promoter selected from the group consisting of PglpF, Plac, PmglB_70UTR, PglpA_70UTR and PglpT_70UTR (SEQ ID NOs: 15, 19, 21, 47 and 48, respectively) and variants thereof, preferably the promoter is a strong promoter selected from the group consisting of SEQ ID NOs 15, 20, 21, 22, 23, 41, 42, 43, 44, 45, 46, 47, 48, 49, 50, 51, and 52.
6. The genetically modified cell according to any one of the preceding items, wherein the cell further comprises a nucleic acid sequence encoding an MFS transporter protein capable of exporting the sialylated HMO into the extracellular medium.
7. The genetically modified cell according to any one of the preceding items, wherein the cell further comprises a nucleic acid sequence encoding a substrate importer capable of importing the initial substrate used for the LST-a production.
8. The genetically modified cell according to item 7, wherein the importer is selected from the group consisting of a lactose permease, a lactose permease mutant according to table 7 and an MFS- or ABC transporter according to table 8.
9. The genetically modified cell according to any one of the preceding items, wherein the cell further comprises a recombinant nucleic acid sequence encoding a β-1,3-galactosyltransferase.
10. The genetically modified cell according to item 9, wherein the genetically modified cell further comprises a recombinant nucleic acid sequence encoding a β-1,3-N-acetyl-glucosaminyltransferase.
11. The genetically modified cell according to any one of items 5 or 10, wherein the β-1,3-N-acetylglucosaminyltransferase is LgtA from Neisseria meningitidis and the β-1,3-galactosyltransferase is GaITK from Helicobacter pylori.
12. The genetically modified cell according to any one of the preceding items, wherein the cell comprises a biosynthetic pathway for making a sialic acid sugar nucleotide.
13. The genetically modified cell according to item 12, wherein the sialic acid sugar nucleotide is CMP-Neu5Ac and said sialic acid sugar nucleotide pathway is encoded by the nucleic acid sequence encoding neuBCA from Campylobacter jejuni (SEQ ID NO: 38).
14. The genetically modified cell according to item 8 or 13, wherein the sialic acid sugar nucleotide pathway is encoded from a high-copy plasmid bearing the neuBCA operon.
15. The genetically modified cell according to any of the preceding items, wherein said modified cell is a microorganism.
16. The genetically modified cell according to any of the preceding items, wherein said modified cell is a bacterium or a fungus.
17. The genetically modified cell according to item 16, wherein said fungus is selected from a yeast cell of the genera Komagataella, Kluyveromyces, Yarrowia, Pichia, Saccaromyces, Schizosaccharomyces or Hansenula or from a filamentous fungous of the genera Aspargillus, Fusarium or Thricoderma.
18. The genetically modified cell according to item 16, wherein said bacterium is selected from the group consisting of Escherichia sp., Bacillus sp., lactobacillus sp. and Campylobacter sp.
19. The genetically modified cell according to item 18 wherein said bacterium is E. coli.
20. A method for producing a sialylated human milk oligosaccharide (HMO), said method comprising culturing a genetically modified cell comprising a recombinant nucleic acid sequence encoding an enzyme with α-2,3-sialyltransferase activity, wherein said enzyme is selected from the group consisting of:
   a. Ccol2 comprising or consisting of the amino acid sequence of SEQ ID NO: 1 or an amino acid sequence with at least 80% identity to SEQ ID NO: 1,
   b. Cjej1 comprising or consisting of the amino acid sequence of SEQ ID NO: 2 or an amino acid sequence with at least 80%, identity to SEQ ID NO: 2,
   c. Csub1 comprising or consisting of the amino acid sequence of SEQ ID NO: 3 or an amino acid sequence with at least 80% identity to SEQ ID NO: 3,
   d. Chepa comprising or consisting of the amino acid sequence of SEQ ID NO: 4 or an amino acid sequence with at least 80% identity to SEQ ID NO: 4, and
   e. Clari1 comprising or consisting of the amino acid sequence of SEQ ID NO: 5 or an amino acid sequence with at least 80% identity to SEQ ID NO: 5; and
   wherein said genetically modified cell optionally comprises at least one additional modification according to any one of items 5 to 14.
21. The method according to item 20, wherein at least 9% of the total molar HMO content produced by said method is LST-a.
22. The method according to any one of items 20 or 21, where the genetically modified cell is a microorganism according to any one of items 16 to 19.
23. The method according to any one of items 20 to 22, wherein the sialylated human milk oligosaccharide (HMO) produced is LST-a and 3′SL.
24. The method according to item 20 to 23, wherein the 3′SL content produced by said cell is below 20% of the total HMO content produced by the cell.
25. The method according to any one of items 20 to 24, wherein the method comprises cultivating the genetically engineered cell in the presence of an energy source selected from the group consisting of glucose, sucrose, fructose, xylose and glycerol.
26. The method according to any one of items 20 to 25, wherein lactose is added during the cultivation of the genetically engineered cells as a substrate for the HMO formation, in particular sialylated HMO.
27. The method according to any one of items 20 to 25, wherein lacto-N-triose (LNT-II) is supplied during the cultivation of the genetically engineered cells as a substrate for the sialylated HMO formation, in particular LST-a.
28. The method according to any one of items 20 to 27 wherein the sialylated human milk oligosaccharide (HMO) is retrieved from the culture medium and/or the genetically modified cell.
29. The method according to item 28, wherein the LST-a is purified to produce at least 75%, such as at least 80%, such as at least 85%, such as at least 90%, such as at least 95% pure LST-a.
30. A nucleic acid construct comprising recombinant nucleic acid sequence encoding an enzyme with α-2,3-sialyltransferase activity, wherein said recombinant nucleic acid sequence is selected from the group consisting of:
   a. Ccol2comprising or consisting of the nucleic acid sequences of SEQ ID NO: 6 or a nucleic acid sequence with at least 80% identity to SEQ ID: 6,
   b. Cjej1 comprising or consisting of the nucleic acid sequences of SEQ ID NO: 7 or a nucleic acid sequence with at least 80% identity to SEQ ID: 7,
   c. Csub1 comprising or consisting of the nucleic acid sequence of SEQ ID NO: 8 or a nucleic acid sequence with at least 80% identity to SEQ ID: 8,
   d. Chepa comprising or consisting of the nucleic acid sequence of SEQ ID NO: 9 or a nucleic acid sequence with at least 80% identity to SEQ ID: 9, and/or
   e. Clari1 comprising or consisting of the nucleic acid sequence of SEQ ID NO: 10 or a nucleic acid sequence with at least 80% identity to SEQ ID: 10; and
   wherein the enzyme encoding sequence is under the control of a promoter sequence selected from the group consisting of PglpF, Plac, PmglB_70UTR, PglpA_70UTR and PglpT_70UTR (SEQ ID NOs: 15, 19, 21, 47 and 48, respectively) and variants thereof.
31. A nucleic acid construct comprising a recombinant nucleic acid sequence encoding an enzyme with α-2,3-sialyltransferase activity according to item 30, for use in a host cell for producing a sialylated HMO, wherein at least 9% of the total molar HMO content produced by the method is LST-a.
32. A genetically modified cell according to any one of items 1 to 19, for use in the production of a sialylated HMO.

### EXAMPLES

### Methods

Unless stated otherwise, standard techniques, vectors, control sequence elements, and other expression system elements known in the field of molecular biology are used for nucleic acid manipulation, transformation, and expression. Such standard techniques, vectors, and elements can be found, e.g., in: Ausubel et al. (eds.), Current Protocols in Molecular Biology (1995) (John Wiley & Sons); Sambrook, Fritsch, & Maniatis (eds.), Molecular Cloning (1989) (Cold Spring Harbor Laboratory Press, NY); Berger & Kimmel, Methods in Enzymology 152: Guide to Molecular Cloning Techniques (1987) (Academic Press); Bukhari et al. (eds.), DNA Insertion Elements, Plasmids and Episomes (1977) (Cold Spring Harbor Laboratory Press, NY); Miller, J. H. Experiments in molecular genetics (1972.) (Cold spring Harbor Laboratory Press, NY)

The embodiments described below are selected to illustrate the invention and are not limiting the invention in any way.

### Enzymes:

28 enzymes were collected following an in-silico selection approach that was based on protein BLAST searches using known α-2,3-sialyltransferases as queries and by exploiting information sources such as scientific articles or databases, e.g., the KEGG and CAZY databases.

TABLE 3
List of the enzymes tested in the framework of the present disclosure

| Enzyme | | | |
|---|---|---|---|
| Name | GenBank ID | Enzyme Length | Origin |
| PM70 | AAK03258.1 | 31 aa C-terminal | Pasteurella multocida subsp. |
| | | deletion | multocida str. Pm70 |
| CstI | AAF13495.1 | 130 aa C-terminal | Campylobacter jejuni |
| | | deletion | |
| CstII | AAF31771.1 | 32 aa C-terminal | Campylobacter jejuni |
| | | deletion | |
| Hcet | WP_014661583.1 | Full Length | Helicobacter cetorum |
| Phkish | WP_065191322.1 | Full Length | Photobacterium kishitanii |
| Pmult | WP_005753497.1 | 24 aa N-terminal | Pasteurella multocida |
| | | deletion | |
| Poral_2 | WP_101774701.1 | 20 aa N-terminal | Pasteurella oralis |
| | | deletion | |
| Neigon | AAW89748.1 | 18 aa N-terminal | Neisseria gonorrhoeae FA 1090 |
| | | deletion | |
| Methasp | MBO7691107.1 | Full Length | Methanobrevibacter sp. |
| Canmeal | WP_164705616.1 | Full Length | Candidatus Methanomethylophilus |
| | | | alvus |
| Gammaba | OUT95537.1 | Full Length | Gammaproteobacteria bacterium |
| | | | TMED36 |
| Ccol | WP_075498955.1 | Full Length | Campylobacter coli |
| Chepa | WP_066776435.1 | Full Length | Campylobacter hepaticus |
| Azos1 | MBS6996416.1 | Full Length | Azospirillum sp. |
| Poral | WP_101774487.1 | Full Length | Pasteurella oralis |
| Cjej1 | EBD1936710.1 | Full Length | Campylobacter jejuni |
| Ccol2 | EAH6554614.1 | Full Length | Campylobacter coli |
| NmN | WP_002244089.1 | Full Length | Neisseria meningitidis |
| CampN | WP_212140471.1 | Full Length | unclassified Campylobacter |
| | | | (multispecies) |
| PmN | WP_005726268.1 | Full Length | Pasteurella (multispecies) |
| MhnNBse | WP_176810284.1 | Full Length | Mannheimia (multispecies) |
| KingN | WP_038313205.1 | Full Length | Kingella kingae |
| GlaeN | WP_111750218.1 | Full Length | Glaesserella (multispecies) |
| Cinf1 | WP_011272254.1 | Full Length | Haemophilus influenzae |
| Csub1 | WP_039664428.1 | Full Length | Campylobacter subantarcticus |
| Clari1 | EGK8106227.1 | Full Length | Campylobacter lari |
| Phkish2 | WP_036792497.1 | 13 aa N-terminal | Photobacterium kishitanii |
| | | deletion | |
| Celter1 | MBD5788313.1 | Full Length | Cellulosimicrobium terreum |

### Strains

The strains (genetically engineered cells) constructed in the present application were based on Escherichia coli K-12 DH1 with the genotype: F⁻, λ⁻, gyrA96, recA1, re/A1, endA1, thi-1, hsdR17, supE44. Additional modifications were made to the E. coli K-12 DH1 strain to generate the MDO strain with the following modifications: lacZ: deletion of 1.5 kbp, lacA: deletion of 0.5 kbp, nanKETA: deletion of 3.3 kbp, me/A: deletion of 0.9 kbp, wcaJ: deletion of 0.5 kbp, mdoH: deletion of 0.5 kbp, and insertion of Plac promoter upstream of the gmd gene.

Methods of inserting gene(s) of interest into the genome of E. coli are well known to the person skilled in the art. Insertion of genetic cassettes into the E. coli chromosome can be done using gene gorging (see e.g., Herring and Blattner 2004 J. Bacteriol. 186: 2673-81 and Warming et al 2005 Nucleic Acids Res. 33(4): e36) with specific selection marker genes and screening methods.

This MDO strain was further engineered to generate an LNT producing strain by chromosomally integrating a beta-1,3-GlcNAc transferase (LgtA from Neisseria meningitidis, homologous to NCBI Accession nr. WP_033911473.1) and a beta-1,3-galactosyltransferase (GaITK from Helicobacter pylori, homologous to GenBank Accession nr. BD182026.1) both under the control of a PglpF promoter, this strain is named the LNT strain.

Codon optimized DNA sequences encoding individual α-2,3-sialyltransferases were genomically integrated into the LNT strain. Additionally, each strain was transformed with a high-copy plasmid bearing the neuBCA operon from Campylobacter jejuni (SEQ ID NO: 38) under the control of the Plac promoter. The neuBCA operon encodes all the enzymes required for the formation of an activated sialic acid sugar nucleotide (CMP-Neu5Ac). CMP-Neu5Ac acts as a donor for the intended glycosyltransferase reaction facilitated by the α-2,3-sialyltransferase under investigation, i.e., the transfer of sialic acid from the activated sugar CMP-Neu5Ac to the terminal galactose of LNT (acceptor) to form LST-a.

The genotypes of the background strain (MDO), LNT strain and the α-2,3-sialyltransferase-expressing strains capable of producing LST-a are provided in Table 4.

TABLE 4
Genotypes of the strains, capable of producing LST-a, used in the present examples.

| | | 2,3-ST cDNA |
|---|---|---|
| Strain ref | Genotype | SEQ ID NO |
| MDO | F-λ- ΔendA1 ΔrecA1 ΔrelA1 ΔgyrA96 Δthi-1 glnV44 | |
| | hsdR17(rk-mK-) ΔlacZ wcaF::Plac ΔnanKETA ΔlacA ΔmelA | |
| | ΔwcaJ ΔmdoH | |
| LNT | MDO, 2x IgtA-PgIpF, 1xgalTK-PgIpF | |
| Ccol2 | LNT, Ccol2-PgIpF, pBS-neuBCA(Plac)-amp | 24 |
| Cjej1 | LNT, Cjej1-PgIpF, pBS-neuBCA(Plac)-amp | 25 |
| Csub1 | LNT, Csub1-PgIpF, pBS-neuBCA(Plac)-amp | 26 |
| Chepa | LNT, Chepa-PgIpF, pBS-neuBCA(Plac)-amp | 27 |
| Clari1 | LNT, Clari1-PgIpF, pBS-neuBCA(Plac)-amp | 28 |
| Ccol | LNT, Ccol-PgIpF, pBS-neuBCA(Plac)-amp | 29 |
| MhnNBse | LNT, MhnNBse-PgIpF, pBS-neuBCA(Plac)-amp | 30 |
| Pmult | LNT, Pmult-PgIpF, pBS-neuBCA(Plac)-amp | 31 |
| Neigon | LNT, Neigon-PgIpF, pBS-neuBCA(Plac)-amp | 32 |
| Poral | LNT, Poral-PgIpF, pBS-neuBCA(Plac)-amp | 33 |
| Cinf1 | LNT, Cinf-PgIpF. pBS-neuBCA(Plac)-amp | 34 |
| PM70 | LNT, PM70 -PgIpF, pBS-neuBCA(Plac)-amp | 35 |
| Cstl | LNT, CstI-PgIpF, pBS-neuBCA(Plac)-amp | 36 |
| Cstll | LNT, CstII-PgIpF, pBS-neuBCA(Plac)-amp | 37 |

*2,3-ST is an abbreviation of alpha-2,3-sialyltransferase, and the sequence is inserted into the genome of the host strain.

### Deep Well Assay

The strains were screened in 96 deep well plates using a 4-day protocol. During the first 24 hours, precultures were grown to high densities and subsequently transferred to a medium that allowed induction of gene expression and product formation. More specifically, during day 1, fresh precultures were prepared using a basal minimal medium supplemented with magnesium sulphate, thiamine and glucose. The precultures were incubated for 24 hours at 34° C. and 1000 rpm shaking and then further transferred to a new basal minimal medium (BMM, pH 7.5) to start the main culture. The new BMM was supplemented with magnesium sulphate, thiamine, a bolus of 20% glucose solution (50 ul per 100 mL) and a bolus of 20% lactose solution (5 ml per 100 ml). Moreover, 50% sucrose solution was provided as carbon source, accompanied by the addition of sucrose hydrolase (invertase), so that glucose was released at a rate suitable for C-limited growth. IPTG (50 mg/ml) was added to induce gene expression and ampicillin antibiotic (100 mg/ml). The main cultures were incubated for 72 hours at 28° C. and 1000 rpm shaking

### Fermentation

The fermentations were carried out in 250 ml fermenters (AMBR 250 Bioreactor system, Sartorius) starting with 100 ml of defined mineral culture medium, consisting of 25 g/L carbon source (glucose), lactose monohydrate, (NH₄)₂HPO₄, KH₂PO₄, MgSO₄×7H₂O, KOH, NaOH, trace element solution, citric acid, antifoam and thiamine. The trace element solution contained Mn, Cu, Fe, Zn as sulfate salts and citric acid. Fermentations were started by inoculation with 2% (v/v) of pre-cultures grown in a similar medium. After depletion of the carbon source contained in the batch medium a sterile feed solution containing glucose, MgSO₄×7H₂O, trace metal solution and anti-foam was fed continuously at a constant feed rate in a carbon-limited manner. Additional lactose was added via bolus additions 20h after feed start and then every 19 h. The pH throughout fermentation was controlled at 6.8 by titration with NH₄OH-solution. Aeration was at 1 VVM using air and dissolved oxygen was controlled above 20% of air saturation.

Throughout the fermentation, samples were taken in order to determine the concentration of HMOs and lactose. Total broth samples were diluted three-fold in deionized water and boiled for 20 minutes. This was followed by centrifugation at 17000 g for 3 minutes, where after the resulting supernatant was analyzed by HPLC. The HPLC measurements were used to accurately calculate the HMO titer, by-product ratios (not shown) and the accumulated yield of HMO on the carbon source. The latter takes also smaller variations in feed rates and dilutions into account and is therefore an important parameter for direct comparison.

### Example 1—In Vivo LST-a Synthesis

Genetically modified cells expressing individual alpha-2,3-sialyltransferase enzymes were screened for their ability to produce the sialylated HMO LST-a.

A group of 28 enzymes (table 3) were compiled for testing their ability to synthesize LST-a when introduced into a genetically modified cells that produce LNT and activated sialic acid (CMP-Neu5Ac).

Genetically modified strains expressing the 28 individual α-2,3-sialyltransferases (table 3) were generated as described in the “Method” section. The cells were screened in a in a fed-batch deep well assay setup as described in the “Method” section. The molar content of individual HMOs produced by the strains was measured by HPLC. In addition, NMR analysis was conducted on the LST-a fraction to confirm that it indeed is LST-a.

Table 4 lists the genotype of the 14 strains that were found to produce LST-a even in very small amounts, the remaining 14 strains tested did not produce any LST-a at all.

The results of the LST-a producing cells are shown in table 5 as the fraction of the total HMO content (in percentage, %) produced by each strain.

TABLE 5
Content of individual HMO's as % of total
HMO content produced by each strain.

| Strain ref. | LNT-II | LNT | 3′SL | LST-a |
|---|---|---|---|---|
| Ccol2 | 4.7 | 63.1 | 10.1 | 22.0 |
| Cjej1 | 4.7 | 67.0 | 10.0 | 18.3 |
| Csub1 | 4.2 | 79.4 | 4.9 | 11.5 |
| Chepa | 5.6 | 65.0 | 17.9 | 11.5 |
| Clari1 | 4.1 | 67.5 | 18.7 | 9.7 |
| CstI | 8.8 | 71.1 | 12.1 | 8.0 |
| CstII | 3.2 | 70.4 | 19.7 | 6.7 |
| Ccol | 5.2 | 81.2 | 10.0 | 3.6 |
| MhnNBse | 4.4 | 78.7 | 13.7 | 3.2 |
| Pmult | 2.4 | 67.3 | 27.2 | 3.0 |
| PM70 | 3.4 | 65.8 | 28.3 | 2.5 |
| Cinf1 | 4.7 | 76.9 | 16.8 | 1.6 |
| Neigon | 3.0 | 74.8 | 20.7 | 1.5 |
| Poral | 2.3 | 64.7 | 31.8 | 1.2 |

No additional HMOs beyond the ones indicated in table 5 were identified in the deep well assay.

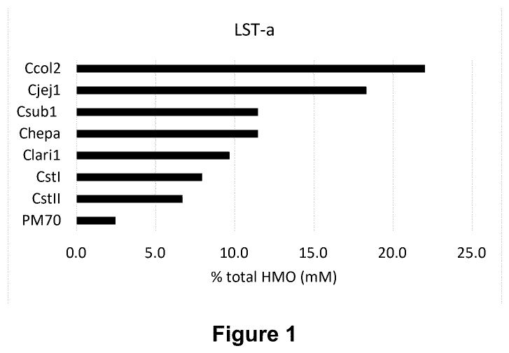

From the data presented in table 5 it can be seen that there are 5 enzymes (Ccol2, Cjej1, Csub1, Chepa, Clari1) that can transfer a sialic acid unit onto the terminal galactose of a LNT molecule to form LST-a at a level above 9% of the total HMO molar content produced by each modified cell which is above the amount of LST-a produced by CstI, CstII and PM70 which are known in the prior art to be active on LNT. The molar % of LST-a produced by these 8 strains is shown in FIG. 1.

Three of the strains, Ccol2, Cjej1 and Csub1, produced almost 2 times more LST-a than 3′SL (which is produced by sialyation of lactose). This indicates that these three enzymes have an increased activity on LNT as substrate contrary lactose as substrate.

Two strains, namely Ccol2 and Cjej1, had the highest LST-a molar content in % of the total amount of HMO produced. Expression of Ccol2 in a strain producing LNT and sialic acid resulted in 22% LST-a. Similarly, expression of the Cjej1 enzyme in a strain producing LNT and sialic acid resulted in 18% LST-a.

### Example 2—Fermentation Using Ccol2, Cjej1, Csub1 and Chepa α-2,3-Sialyltransferase Strains

To confirm the high level of LST-a observed in the deep well assays of Ccol2, Cjej1 Csub1 and Chepa strains of example 1, the four strains were fermented as described in the “Method” section above.

The results are shown in table 6.

TABLE 6
Content of individual HMO's as % of
total HMO content produced by each strain

| Strain ref. | pLNH2 | LNT-II | LNT | 3′SL | LST-a |
|---|---|---|---|---|---|
| Ccol2 | 0 | 7 | 51 | 8 | 33 |
| Cjej1 | 1 | 10 | 56 | 9 | 26 |
| Csub1 | 1 | 11 | 74 | 3 | 11 |
| Chepa | 1 | 21 | 64 | 7 | 9 |

From the data presented table 6, it can be seen that the molar fraction LST-a of the total amount of HMO produced by Ccol2 and Cjej1 strains was higher when the culturing was done in fermenters compared to the Deep well assays of example 1, showing the ability of these strains to produce LST-a at a level above 20% of the total HMO produced by these strains.

The Csub1, Ccol2 and Cjej1 strains also seem to maintain the beneficial ratio above 2:1 of LST-a:3′SL, and all the way up to a 4:1 LST-a:3′SL ratio for Ccol2 and close to 3:1 for the two Csub1 and Cjej1 strains. The Chepa strain also has an improved LST-a:3′SL ratio in the fermentation, which is close to 1:1 compared to the 1:1.5 observed in example 1.

1. A genetically modified cell comprising a recombinant nucleic acid sequence encoding an enzyme with α-2,3-sialyltransferase activity, which is capable of producing at least 9% LST-a of the total molar HMO content produced by the cell.

2. The genetically modified cell according to claim 1, wherein said α-2,3-sialyltransferase enzyme is selected from the group consisting of:
a. Ccol2 comprising the amino acid sequence of SEQ ID NO: 1 or an amino acid sequence with at least 80% identity to SEQ ID NO: 1,
b. Cjej1 comprising the amino acid sequence of SEQ ID NO: 2 or an amino acid sequence with at least 80% identity to SEQ ID NO: 2,
c. Csub1 comprising the amino acid sequence of SEQ ID NO: 3 or an amino acid sequence with at least 80% identity to SEQ ID NO: 3,
d. Chepa comprising the amino acid sequence of SEQ ID NO: 4 or an amino acid sequence with at least 80% identity to SEQ ID NO: 4, and
e. Clari1 comprising the amino acid sequence of SEQ ID NO: 5 or an amino acid sequence with at least 80% identity to SEQ ID NO: 5.

3. The genetically modified cell according claim 1, wherein the cell is further capable of producing 3′SL.

4. The genetically modified cell according to claim 3, wherein the 3′SL produced does not exceed 20% of the total molar content of the HMOs produced by the cell.

5. The genetically modified cell according to claim 1, wherein the cell further comprises a recombinant nucleic acid sequence encoding a β-1,3-galactosyltransferase.

6. The genetically modified cell according to claim 5, wherein the genetically modified cell further comprises a recombinant nucleic acid sequence encoding a β-1,3-N-acetyl-glucosaminyltransferase.

7. The genetically modified cell according to claim 6, wherein the β-1,3-N-acetylglucosaminyltransferase is LgtA from Neisseria meningitidis and the β-1,3-galactosyltransferase is GalTK from Helicobacter pylori.

8. The genetically modified cell according to claim 1, wherein the cell comprises a biosynthetic pathway for making a sialic acid sugar nucleotide.

9. The genetically modified cell according to claim 8, wherein the sialic acid sugar nucleotide is CMP-Neu5Ac and the sialic acid sugar nucleotide pathway is encoded by the nucleic acid sequence encoding neuBCA from Campylobacter jejuni (SEQ ID NO: 38).

10. (canceled)

11. The genetically modified cell according to claim 1, wherein the cell is selected from the group consisting of Escherichia Coli, Bacillus subtilis, lactobacillus lactis, Corynebacterium glutamicum, Yarrowia lipolytica, Pichia pastoris, and Saccharomyces cerevisiae.

12. (canceled)

13. A method for producing a sialylated human milk oligosaccharide (HMO), comprising culturing a genetically modified cell comprising a recombinant nucleic acid sequence encoding an enzyme with α-2,3-sialyltransferase activity, wherein the enzyme is selected from the group consisting of:
a. Ccol2 comprising or consisting of the amino acid sequence of SEQ ID NO: 1 or an amino acid sequence with at least 80% identity to SEQ ID NO: 1,
b. Cjej1 comprising or consisting of the amino acid sequence of SEQ ID NO: 2 or an amino acid sequence with at least 80%, identity to SEQ ID NO: 2,
c. Csub1 comprising or consisting of the amino acid sequence of SEQ ID NO: 3 or an amino acid sequence with at least 80% identity to SEQ ID NO: 3,
d. Chepa comprising or consisting of the amino acid sequence of SEQ ID NO: 4 or an amino acid sequence with at least 80% identity to SEQ ID NO: 4, and
e. Clari1 comprising or consisting of the amino acid sequence of SEQ ID NO: 5 or an amino acid sequence with at least 80% identity to SEQ ID NO: 5.

14. The method according to claim 13, wherein at least 9% of the total molar HMO content produced by the method is LST-a.

15. The method according to claim 14, where the genetically modified cell is E. coli.

16. The method according to claim 13, wherein the sialylated human milk oligosaccharide (HMO) produced is LST-a and 3′SL.

17. The method according to claim 13, wherein the 3′SL content produced by the cell is below 20% of the total HMO content produced by the cell.

18. The method according to claim 13, wherein the method further comprises cultivating the genetically engineered cell in the presence of an energy source selected from the group consisting of glucose, sucrose, fructose, xylose and glycerol.

19. The method according to claim 18, wherein lactose is added during the cultivation of the genetically engineered cell.

20. The method according to claim 18, wherein lacto-N-triose (LNT-II) is supplied during the cultivation of the genetically engineered cell.

21. The method according to claim 14, wherein the method further comprises retrieving the sialyated HMO from the culture medium and/or the genetically modified cell.

22. The method according to claim 21, wherein the LST-a is purified to produce at least 75% pure LST-a.

23-25. (canceled)


SEQUENCE LISTING (56 sequences)

SEQ ID NO: 1 - protein, 292 aa, Campylobacter coli
MKKVIIAGNGPSLKEIDYSRLPNDFDVFRCNQFYFEDKYYLGKKCKAVFYNPSLFFEQYYTLKHLIQNQEYETELIVCSNFNLTHIESENFLKNFYDYFPDAHLGYDFFKQLKEFNAYFKFHEIYFNQRITSGIYMCAVAIALGYKEIYLSGIDFYQNGSSYAFDTKQKNLLKLVSNFKNDNSHYIGHSKNTDLKALEFLEKTYEIKLYCLCPNSLLANFIELAPNLNSNFIIQEKNNYIKDILIPSKEAYNNFSKNFNLPNKPKLKENIYFKILQDLLKLPSDIKHYFKGK

SEQ ID NO: 2 - protein, 289 aa, Campylobacter jejuni
MKKAIIAGNAPSLKNIDYTLLPQDYDVFRCNQFYLEDKYYLGKKLKAVFFNSCVFFENYYTLKELIKNEEYTTSLIFCSSHKHLEEKDFLENFKDFYPDSTMGHEILSRLEKFYAWTIFNDVYKNRRFTSAIYMCAIAVAMGYKELYLAGIDFYNTGSTYAYTQGENLTRIFGHFKKYNGHTQDIELEALELLKELYDVKIYCLCPTSPLAKFVTPPPSTSHLNHFILENKPQNHINDMLLPSKRAYKNARKLRNYDNSKLTDNLYYRLIKDLIKLPSNIKHHLKTRRF

SEQ ID NO: 3 - protein, 300 aa, Campylobacter subantarcticus
MQGGGNAIICGNGPSLKDIDYKKLPRKYDVFRCNQFYFEEKYYLGKNIKYAFFNPFVFFEQYYTTKKLIEKKEYNIENIICSTFNLPTVDNENFIKNFNNYFCDAYLGHEILHNIKDFLAFIKYNELYENNRITSGIYMCAIAIALGYKNIYLCGIDFYNDKNNMYGFDNKKENLLKLNPEFLKKDSVYTKHSKEFDLKALEFLKEIYGVNFYSLNNNELSKYIPLAPNTNNNFVLIDKDKNYVNDILIPKEKDYCKLFKQEDGKIKLKENVYYKLFKDLFRLPSDIKHYLKEKYANKNR

SEQ ID NO: 4 - protein, 296 aa, Campylobacter hepaticus
MRKAIIAGNGPSLKEIDYTKLPIDYDVFRCNQFYFEDKYYLGKNCKAVFYNPSLFFEQYYTLKHLIDKKEYKTDFIFCSTFNLVHLENENFSKIFYNYFPDAHLGYDFLKTLKEFDAYCKFHEIYLNQRITSGIYMCAIAIALGYKEIYLAGIDFYHNGSFYAFNTKQNNLIKLLPNFKNDNSHNIKHTKNMDIKALEFLEKTYEVQFYCLCPNSPLSHFIKTPPPVKNSTFKLEEKSNYIKDILIPSKEAYDIFSINFNVSKKPRLKQNIYYRLIENLLKLPSDIKHYYKSRKLK

SEQ ID NO: 5 - protein, 301 aa, Campylobacter lari
MVGGGNAVICGNGPSLKNIDYKRLPKEFDVFKCNQFYFEDRYFVGKNIKYAFFNPFVFFEQYYTSKKLIQNGEYIIENIVCSTFNLPLIDNENFIKLFPSYFCDALLGHEVLAKINDFFAFVKYNEIYENKRITSGIYMCAAAVALGYKNIYLTGIDFYDDKNNMYAFESKQHNILSLLPNFKNKDSVYEAHSKNFDLETLIFLKEKYNVNFYALNENSPISKYIDLAPIENSNFILKDKPSNYINDILIPNSKYKNTIYKVQNQDSKLKQNIYYKLFKDLFHLPSDIKHYLKEKYANKNR

SEQ ID NO: 6 - protein, 302 aa, Campylobacter coli
MQNVIIAGNGPSLQSINYQRLPKEYDIFRCNQFYFEDKYYLGKNIKAAFFNPYPFLQQYHTAKQLVFNNEYKIENIFCSTFNLPFIEKDNFINKFYDFFPDAKLGHKIIENLKEFYAYIKYNEIYLNKRITSGIYMCAIAIALGYKNIYLCGIDFYEGETIYPFKAMSKNIKKIFPWIKDFNPSNFHSKEYDIEILKLLESIYKVNIYALCDNSALANYFPLLVNTDNSFVLENKSDDCINDILLTNNTPGINFYKSQIQVNNTEILLLNFQNMISAKENEISNLNKILQDSYKTINTKENE

SEQ ID NO: 7 - protein, 312 aa, Mannheimia sp.
MNLIICCTPLQVLIAEKILEIHRNEQFFGVMLSTVKNAKFDFYQSRLAKKCQQFFAMQQHSERIQLLKEIIYLKSTFGGKKFNKVFLANINELEIQFLLSAINFNEINTFDDGIANIVQSGIFYKQECSGINRKLINTFLGNRYSLEKLKKLSKKHYTIYKGFPNIIENTVYIELINSTTLPDSELNNSEVVNILLGQPIFERDDEKNIILAEKVIRQFNIDRYFPHPREKYQISNVEYIKTNLILEDYLFQECSDKKCRVYTYFSSAVINILNKSPNIEVVALRVNVDNPTYIESYVLLENLGINIIDIRE

SEQ ID NO: 8 - protein, 388 aa, Pasteurella multocida
MKTITLYLDPASLPALNQLMDFTQNNEDKTHPRIFGLSRFKIPDNIITQYQNIHFVELKDNRPTEALFTILDQYPGNIELNIHLNIAHSVQLIRPILAYRFKHLDRVSIQQLNLYDDGSMEYVDLEKEENKDISAEIKQAEKQLSHYLLTGKIKFDNPTIARYVWQSAFPVKYHFLSTDYFEKAEFLQPLKEYLAENYQKMDWTAYQQLTPEQQAFYLTLVGFNDEVKQSLEVQQAKFIFTGTTTWEGNTDVREYYAQQQLNLLNHFTQAEGDLFIGDHYKIYFKGHPRGGEINDYILNNAKNITNIPANISFEVLMMTGLLPDKVGGVASSLYFSLPKEKISHIIFTSNKQVKSKEDALNNPYVKVMRRLGIIDESQVIFWDSLKQL

SEQ ID NO: 9 - protein, 360 aa, Neisseria gonorrhoeae
MIFYTFDRVNQGERNAVSLLKDKLFNEEGKPVNLIFCYTILQMKVAERIMAQHPGERFYVVLMSENRNEKYDYYFNQIKDKAERAYFFYLPYGLNKSFNFIPTMAELKVKSMLLPKVKRIYLASLEKVSIAAFLSTYPDAEIKTFDDGTNNLIRESSYLGGEFAVNGAIKRNFARMMVGDWSIAKTRNASDEHYTIFKGLKNIMDDGRRKMTYLPLFDASELKAGDETGGTVRILLGSPDKEMKEISEKAAKNFNIQYVAPHPRQTYGLSGVTALNSPYVIEDYILREIKKNPHTRYEIYTFFSGAALTMKDFPNVHVYALKPASLPEDYWLKPVYALFRQADIPILAFDDKNQSHGKSK

SEQ ID NO: 10 - protein, 303 aa, Pasteurella oralis
MDEVERSRECKSVVVAGNGASLTQVDYSLLPVNYDVFRCNQFYFEDHYFLGKKIKAAFFTPGVLLEQYYTLYHLRKNNEYSVDKIILSSFNHPTVDLSKSENIKKLFIDVINGYEEYLSKLSDFDIYLRYKELYESQRITSGIYMCAVAIAMGYTDIYLTGIDFYESNSKSYAFEPKKTNIISLLPDFKNISSKFHYHNKDADLEALYFLQKNYSVNFYSISPGSLLYEHFPTSTKNISSQTNLIPVKKENYINDILLPPDFVYEKLGFVNSKKERLNANLIFRIIKDFIKLPSAIKHYLREK

SEQ ID NO: 11 - protein, 326 aa, Haemophilus influenzae
MNGTICPINQSINQSINQSINQSINQSINQSKSVIIAGNGTSLKSIDYSLLPKDYDVFRCNQFYFEDHYFLGKKIKKVFFNCSVIFEQYYTFMQLIKNNEYEYADIILSSFLNLGDSELKKIQRLEKLLPQIDLGHSYLKKLRAFDAHLQYHELYENKRITSGVYMCAVATAMGYKDLYLTGIDFYQEKGNPYAFHHQTENIIKLLPSFSQNKSQSDIHSMEYDLNALYFLQKHYGVNIYCISPESPLCNYFPLSPLNNPITFILEEKKNYTQDILIPPKFVYKKIGIYSKPRIYQNLIFRLIWDILRLPNDIKHALKAKKMRLRK

SEQ ID NO: 12 - protein, 272 aa, Pasteurella multocida
MDKFAEHEIPKAVIVAGNGESLSQIDYRLLPKNYDVFRCNQFYFEERYFLGNKIKAVFFTPGVFLEQYYTLYHLKRNNEYFVDNVILSSFNHPTVDLEKSQKIQALFIDVINGYEKYLSKLTAFDVYLRYKELYENQRITSGVYMCAVAIAMGYTDIYLTGIDFYQASEENYAFDNKKPNIIRLLPDFRKEKTLFSYHSKDIDLEALSFLQQHYHVNFYSISPMSPLSKHFPIPTVEDDCETTFVAPLKENYINDILLPPHFVYEKLGTIVS

SEQ ID NO: 13 - protein, 259 aa, Campylobacter jejuni
MKKVIIAGNGPSLKEIDYSRLPNDFDVFRCNQFYFEDKYYLGKKCKAVFYNPILFFEQYYTLKHLIQNQEYETELIMCSNYNQAHLENENFVKTFYDYFPDAHLGYDFFKQLKDFNAYFKFHEIYFNQRITSGVYMCAVAIALGYKEIYLSGIDFYQNGSSYAFDTKQKNLLKLAPNFKNDNSHYIGHSKNTDIKALEFLEKTYKIKLYCLCPNSLLANFIELAPNLNSNFIIQEKNNYTKDILIPSSEAYGKFSKNIN

SEQ ID NO: 14 - protein, 259 aa, Campylobacter jejuni; identical to SEQ ID NO: 13

SEQ ID NO: 15 - DNA, 300 bp, synthetic construct
  misc_feature 1..300: note = Promoter Sequence
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaggaggaaacagct

SEQ ID NO: 16 - DNA, 300 bp, synthetic construct
  misc_feature 1..300: note = Promoter sequence
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaattcgaaacagct

SEQ ID NO: 17 - DNA, 300 bp, synthetic construct
  misc_feature 1..300: note = Promoter sequence
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagaacaaaacagct

SEQ ID NO: 18 - DNA, 300 bp, synthetic construct
  misc_feature 1..300: note = Promoter sequence
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagcaaaacagct

SEQ ID NO: 19 - DNA, 107 bp, Escherichia coli
  source 1..107: mol_type = other DNA
tgtgagttagctcactcattaggcaccccaggctttacactttatgcttccggctcgtatgttgtgtggaattgtgagcggataacaatttcacacaggaaacagct

SEQ ID NO: 20 - DNA, 141 bp, synthetic construct
  misc_feature 1..141: note = Promoter sequence
  source 1..141: mol_type = other DNA
tgtgagttagctcactcattaggcaccccaggctttacactttatgcttccggctcgtatgttgtgtggaatgcctacaagcatcgtggaggtccgtgactttcacgcatacaacaaacattaaccaaggaggaaacagct

SEQ ID NO: 21 - DNA, 203 bp, synthetic construct
  misc_feature 1..203: note = Promoter sequence
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaggaggaaacagct

SEQ ID NO: 22 - DNA, 203 bp, synthetic construct
  misc_feature 1..203: note = Promoter sequence
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaactaggaaacagct

SEQ ID NO: 23 - DNA, 203 bp, synthetic construct
  misc_feature 1..203: note = Promoter sequence
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaagagaaaaacagct

SEQ ID NO: 24 - DNA, 879 bp, Campylobacter coli
  source 1..879: mol_type = other DNA
atgaagaaggtgattattgcaggtaatggtccgagcctgaaagaaattgattatagccgtctgccgaacgattttgatgtttttcgttgcaaccagttctacttcgaggataaatactatctgggcaaaaagtgcaaagccgtgttttataacccgagcctgtttttcgaacagtactataccctgaaacatctgatccagaatcaagagtatgaaaccgaactgattgtgtgcagcaactttaatctgacccatatcgaaagcgagaacttcctgaaaaacttctatgattattttccggatgcgcacctgggctatgatttctttaaacagctgaaagagttcaacgcctacttcaaatttcacgagatctattttaaccagcgtattaccagcggcatttatatgtgtgcagttgcaattgcgctgggctataaagaaatttatctgagcggcatcgatttctatcagaatggtagcagctatgcctttgacaccaaacagaaaaatctgctgaaactggtgagcaacttcaaaaacgataacagccattatatcggccacagcaaaaataccgatctgaaagcactggaatttctggaaaagacctatgagatcaaactgtattgtctgtgtccgaatagcctgctggcaaactttattgaactggcaccgaatctgaactccaactttattatccaagagaaaaacaactacatcaaagatattctgatcccgagcaaagaggcctataacaactttagcaaaaacttcaacctgccgaacaaaccgaaactgaaagagaacatctatttcaaaattctgcaggacctgctgaaattgccgagcgatatcaaacattacttcaagggcaaataa

SEQ ID NO: 25 - DNA, 870 bp, Campylobacter jejuni
  source 1..870: mol_type = other DNA
atgaagaaagccattattgcaggtaatgcaccgagcctgaaaaacattgattataccctgctgccgcaggattatgatgtttttcgttgcaatcagttctatctggaagataaatactacctgggcaaaaagctgaaagccgtgtttttcaatagctgcgtgttcttcgaaaactattacaccctgaaagagctgatcaagaacgaagaatataccacgagcctgatcttttgtagcagccataaacacctggaagagaaagattttctggaaaacttcaaagacttctatccggattcaaccatgggtcatgaaattctgagccgtctggaaaaattctatgcatggaccattttcaacgatgtgtataaaaaccgtcgttttaccagcgccatttatatgtgtgcaattgccgttgcaatgggctataaagaactgtatctggcaggcatcgatttctataataccggtagcacctatgcatatacccagggtgaaaatctgacccgtatttttggtcacttcaagaaatataacggccacacgcaggatattgaactggaagcactggaactgctgaaagaattatatgacgtgaagatctattgtctgtgtccgaccagtccgctggcaaaatttgttacaccgcctccgagcaccagccatctgaatcattttatcctggaaaataaaccgcagaaccacatcaatgatatgctgctgccgagcaaacgtgcctataaaaacgcacgtaaactgcgcaattatgacaatagcaaactgaccgataacctgtattatcgcctgatcaaagacctgattaaactgccgagtaacatcaaacatcatctgaaaacccgtcgcttctaa

SEQ ID NO: 26 - DNA, 903 bp, Campylobacter subantarcticus
  source 1..903: mol_type = other DNA
atgcaaggtggtggtaatgccattatttgtggtaatggtccgagcctgaaagatatcgattataagaaactgcctcgcaagtatgatgtgtttcgttgcaatcagttctacttcgaggaaaaatactacctgggcaagaacatcaaatacgcctttttcaatccgttcgtgttcttcgaacagtattacaccaccaaaaagctgatcgagaagaaagagtataacatcgagaacattatctgcagcacctttaatctgccgaccgtggataatgaaaacttcatcaaaaacttcaacaactatttttgcgacgcctatctgggtcatgaaattctgcataacatcaaagattttctggcctttatcaaatacaacgaactgtatgaaaacaaccgtattaccagcggcatttatatgtgtgcaattgccattgcgctgggctataaaaacatttatctgtgcggcatcgatttctataacgataaaaacaacatgtacggcttcgacaacaagaaagaaaatctgctgaaactgaacccggaattcctgaaaaaggatagcgtttataccaagcacagcaaagagtttgatctgaaagcactggaatttctgaaagaaatctatggcgtgaacttctacagcctgaataataacgaactgagcaaatatatcccgctggcaccgaataccaataataactttgtgctgatcgacaaagataaaaactatgtgaacgacattctgatcccgaaagagaaagattactgcaagctgttcaaacaagaggacggtaaaatcaagctgaaagagaacgtctactataaactgttcaaagacctgtttcgtctgccgagcgatattaaacattaccttaaagagaagtacgccaacaagaaccgctaa

SEQ ID NO: 27 - DNA, 891 bp, Campylobacter hepaticus
  source 1..891: mol_type = other DNA
atgcgtaaagcaattattgcaggtaatggtccgagcctgaaagaaatcgattataccaaactgccgatcgattatgatgtgtttcgttgcaaccagttctatttcgaggataaatactacctgggcaaaaattgcaaagccgtgttttataacccgagcctgtttttcgaacagtactataccctgaaacacctgatcgacaaaaaagagtataagaccgatttcatcttctgcagcacctttaatctggtgcatctggaaaatgagaactttagcaagatcttctacaactattttccggatgctcatctgggctatgattttctgaaaacgctgaaagaatttgatgcctactgcaaattccacgagatttatctgaatcagcgtattaccagcggcatttatatgtgtgcaattgccattgcgctgggctataaagaaatttatctggcaggcatcgacttctatcataacggtagcttttatgccttcaacaccaaacagaacaacctgattaaactgctgccgaacttcaaaaacgataacagccataacatcaaacacaccaagaacatggatatcaaagccctggaatttctggaaaaaacctatgaggttcagttctactgtctgtgtccgaatagtccgctgagccatttcattaaaacaccgcctccggtgaaaaatagcacgtttaaactggaagagaaaagcaactacatcaaagacattctgatcccgagcaaagaagcctacgatatctttagcattaactttaacgtgagcaagaaaccgcgtctgaaacagaatatctattatcgcctgattgagaacctgctgaaactgccgtcagatatcaaacactattacaaaagccgcaaactgaaataa

SEQ ID NO: 28 - DNA, 906 bp, Campylobacter lari
  source 1..906: mol_type = other DNA
atggttggtggtggtaatgcagttatttgtggtaatggtccgagcctgaaaaacatcgattataaacgtctgccgaaagaattcgacgtgtttaaatgcaaccagttctatttcgaggatcgctattttgtgggcaagaacatcaaatatgcctttttcaatccgttcgtgttcttcgagcagtattataccagcaaaaagctgattcagaacggcgaatatatcatcgaaaatattgtgtgcagcacctttaatctgccgctgattgataacgagaacttcattaaactgttcccgagctatttttgtgatgcactgctgggtcatgaagtgctggccaaaatcaatgatttctttgccttcgtgaagtacaacgagatctatgaaaataaacgtatcaccagcggcatctatatgtgtgccgcagcagttgcactgggctataaaaacatttatctgaccggcatcgatttctacgacgacaaaaataacatgtatgccttcgaaagcaagcagcataatattctgagcctgctgccgaacttcaaaaacaaagatagcgtttatgaagcccacagcaaaaactttgatctggaaaccctgatctttctgaaagaaaaatacaacgtgaacttttatgccctgaatgaaaatagcccgatcagcaaatatatcgatctggcaccgattgaaaactccaactttatcctgaaagataaaccgagcaactacatcaacgatattctgattccgaacagcaagtacaagaacaccatctataaagtgcagaaccaggacagcaaactgaaacagaacatctactacaaactgttcaaggacctgtttcatctgccgagcgatattaaacattatcttaaagagaaatacgccaacaaaaatcgctaa

SEQ ID NO: 29 - DNA, 909 bp, Campylobacter coli
  source 1..909: mol_type = other DNA
atgcagaatgttattattgcaggtaatggtccgagcctgcagagcattaactatcagcgtctgccgaaagaatatgatattttccgttgcaaccagttctacttcgaggataaatactatctgggcaagaacattaaagcagccttttttaacccgtatccgtttctgcagcagtatcataccgcaaaacagctggtttttaacaacgagtacaagatcgaaaacatcttttgcagcacctttaacctgccgtttatcgagaaagacaacttcatcaacaagttctatgatttcttcccggatgcaaaactgggccataaaatcattgaaaacctgaaagagttctacgcctacatcaaatacaacgagatctatctgaacaaacgtattaccagcggcatttatatgtgtgcaattgccattgcgctgggctataaaaacatctatctgtgcggcatcgatttctatgaaggcgaaaccatttatccgtttaaagccatgagcaaaaacatcaaaaaaatctttccgtggatcaaagacttcaacccgagcaattttcacagcaaagagtatgacatcgagatcctgaaactgctggaaagcatctataaagtgaacatttatgccctgtgcgataatagcgcactggcaaactattttccgctgctggttaatacggataatagctttgttctggaaaacaaaagcgacgattgcatcaatgatattctgctgaccaataatacccctggcatcaacttctataaaagccagattcaggtgaacaacaccgaaattctgctgctgaattttcagaacatgatcagcgccaaagaaaacgaaattagcaacctgaacaaaattctgcaggacagctacaaaaccatcaacaccaaagagaacgagtaa

SEQ ID NO: 30 - DNA, 939 bp, Mannheimia sp.
  source 1..939: mol_type = other DNA
atgaacctgattatttgttgtacaccgctgcaggttctgattgcagagaaaattctggaaattcaccgcaacgaacagtttttcggtgttatgctgagcaccgttaaaaacgccaaattcgatttttatcagagccgtctggcaaagaaatgtcagcagttctttgcaatgcagcagcatagcgaacgtattcagctgctgaaagaaatcatctatctgaaaagcaccttcggtggcaaaaagttcaataaagtttttctggccaacatcaacgagctggaaatccagtttctgctgagcgcaattaactttaacgaaatcaacaccttcgatgacggcattgccaatattgttcagagcggcattttctacaaacaagaatgcagcggtattaaccgcaaactgattaatacctttctgggcaatcgttacagcctggaaaaactgaaaaagctgagcaaaaagcactataccatctataaaggcttcccgaacattatcgaaaacaccgtgtatatcgaactgatcaatagcaccacactgccggatagcgaactgaataatagcgaagttgttaatattctgctgggccagccgatttttgaacgtgatgatgagaaaaacattatcctggccgaaaaagtgattcgccagtttaacattgatcgctattttccgcatccgcgtgaaaaatatcagattagcaatgtcgagtatatcaaaaccaacctgatcctggaagattacctgtttcaagaatgtagcgataaaaagtgccgtgtgtatacctattttagcagcgcagttattaacatcctgaacaaaagcccgaatattgaagttgttgcactgcgtgttaatgtggataatccgacctatattgaaagctatgtgctgctggaaaatctgggcattaacattattgatatccgcgaataa

SEQ ID NO: 31 - DNA, 1167 bp, Pasteurella multocida
  source 1..1167: mol_type = other DNA
atgaaaaccattacgctgtatttagatccggcaagcctgcctgcactgaaccagctgatggattttacccagaataacgaggataaaacccatccgcgtatctttggtctgagccgctttaaaatcccggataacattattacccagtaccagaacatccacttcgtggaactgaaagataatcgtccgaccgaagcactgtttaccattctggatcagtatccgggtaatatcgaactgaacatccatctgaatattgcccatagcgttcagctgattcgtccgattctggcatatcgttttaaacatctggatcgtgttagcattcagcagctgaacctgtatgatgatggtagcatggaatatgtggatctggaaaaagaagagaacaaagatatcagcgcagaaatcaaacaggcagaaaaacagctgagccattatctgctgaccggcaaaattaagtttgataatccgaccattgcgcgttatgtttggcagagcgcatttccggttaaatatcattttctgagcaccgactatttcgagaaagcagaatttctgcagccgctgaaagaatatctggcagaaaattaccagaaaatggattggaccgcatatcagcaactgacaccggaacagcaggcattttatctgaccctggttggttttaacgatgaagttaaacagagcctggaagttcagcaggccaaatttatctttaccggcaccaccacctgggaaggtaataccgatgttcgtgaatattatgcacagcaacagctgaatctgctgaatcattttacacaggccgaaggtgacctgtttattggtgatcattacaaaatctatttcaaaggtcatccgcgtggtggcgaaattaatgattatattctgaacaacgccaaaaacatcaccaacattccggcaaacattagctttgaagttctgatgatgaccggtctgctgccggataaagttggtggtgttgcaagcagcctgtattttagcctgccgaaagaaaaaatcagccacatcattttcaccagcaacaaacaggtgaaaagcaaagaagatgcactgaataacccgtacgttaaagttatgcgtcgtctgggtattattgatgaaagccaggttatcttttgggacagcctgaaacagctgtaa

SEQ ID NO: 32 - DNA, 1080 bp, Neisseria gonorrhoeae
  source 1..1080: mol_type = other DNA
atgatcttctatacctttgatcgcgtgaatcagggtgaacgtaatgcagttagcctgctgaaagataaactgtttaacgaagaaggcaaaccggtcaatctgatcttttgttataccatcctgcagatgaaagtggccgaacgtattatggcacagcatccgggtgaacgcttttatgttgttctgatgagcgaaaaccgcaacgagaaatatgattactactttaaccagatcaaggacaaagcagaacgtgcctatttcttttatctgccgtatggtctgaacaaaagctttaactttattccgaccatggccgagctgaaagttaaaagcatgctgctgccgaaagtgaaacgtatttatctggcaagcctggaaaaagttagcattgcagcatttctgagcacctatccggatgcagaaatcaaaacctttgatgatggcaccaataacctgattcgtgaaagcagctatttaggtggtgaatttgcagttaatggtgcgatcaaacgtaattttgcacgtatgatggttggcgattggagcattgcaaaaacccgtaatgcatccgatgaacactataccatctttaaaggcctgaagaacattatggatgatggtcgtcgtaaaatgacctatctgccgctgtttgatgcaagcgaactgaaagccggtgatgaaaccggtggcaccgttcgtattctgctgggtagtccggataaagaaatgaaagaaattagcgagaaagcagccaaaaacttcaacattcagtatgttgcaccgcatccgcgtcagacctatggtctgtcaggtgttaccgcactgaatagcccgtatgtgattgaagattatattctgcgcgagatcaaaaagaatccgcatacacgttatgagatctacacctttttcagcggtgcagcactgaccatgaaagattttccgaatgttcatgtgtatgcactgaaaccggcaagtctgccggaagattattggctgaaacctgtttatgcactgtttcgtcaggcagatattccgattctggcattcgatgataaaaatcagagccacggtaaaagcaaa

SEQ ID NO: 33 - DNA, 912 bp, Pasteurella oralis
  source 1..912: mol_type = other DNA
atggatgaagttgaacgtagccgtgaatgtaaaagcgttgttgttgcaggtaatggtgcaagtctgacccaggttgattatagcctgctgccggttaattatgatgtgtttcgttgcaaccagttctatttcgaggatcattattttctgggcaaaaagatcaaagcagccttttttacaccgggtgttctgctggaacagtattacaccctgtatcatctgcgcaaaaacaacgaatatagcgtggataaaatcatcctgagcagctttaatcatccgaccgttgatctgagcaaaagcgagaatatcaaaaagctgttcatcgatgtgatcaacggctatgaagaatacctgagcaaactgagcgatttcgatatttatctgcgctacaaagaactgtatgaaagccagcgtattaccagcggcatttatatgtgtgcagttgcaattgccatgggctataccgatatctatctgaccggtatcgatttctatgagagcaacagcaaaagctatgcctttgaaccgaaaaagaccaacattattagtctgctgcccgactttaaaaacatcagcagcaaatttcactaccacaacaaagatgcagatctggaagcactgtattttctgcaaaaaaactacagcgtgaacttttacagcattagtccgggttcactgctgtatgaacattttccgaccagcaccaaaaacattagctcacagaccaatctgattccggtgaagaaagagaactatatcaacgatattctgctgcctccggatttcgtttatgaaaaactgggttttgtgaatagcaaaaaagaacgcctgaatgccaacctgattttccgcattatcaaagactttatcaaactgccgagcgccatcaaacattatctgcgtgaaaaataa

SEQ ID NO: 34 - DNA, 981 bp, Haemophilus influenzae
  source 1..981: mol_type = other DNA
atgaacggcaccatttgtccgattaatcagagcattaaccagtccatcaaccagagcatcaatcaatccattaatcagtcaattaatcaaagcaaaagcgtgatcattgcaggtaatggcaccagcctgaaaagcattgattatagcctgctgccgaaagattatgatgtgtttcgttgcaaccagttctatttcgaggatcattattttctgggcaaaaagatcaaaaaggtgttctttaactgcagcgtgatcttcgaacagtattatacctttatgcagctgatcaaaaacaacgagtatgagtatgccgatattatcctgagcagctttctgaatctgggtgatagcgaactgaaaaagattcagcgtctggaaaaactgctgccgcagattgatctgggtcatagttatctgaagaaactgcgtgcatttgatgcacatctgcagtatcatgaactgtatgaaaacaaacgtattaccagcggtgtttatatgtgtgcagttgcaaccgcaatgggttataaagatctgtatctgaccggcatcgatttctatcaagaaaaaggtaatccgtatgcctttcatcatcagaccgaaaacattatcaaactgttaccgagcttcagccagaataaaagccagagcgatattcacagcatggaatatgatctgaacgccctgtatttcctgcagaaacattatggcgttaacatctattgcatcagtccggaaagtccgctgtgtaactattttccgctgagtccgctgaataatccgattaccttcattctggaagagaaaaagaactacacccaggatattctgatccctccgaaattcgtgtataaaaagattggcatctatagcaagcctcgcatttatcagaatctgatttttcgcctgatttgggatatcctgcgtctgccgaatgatattaaacatgcactgaaagccaaaaagatgcgcctgcgtaaataa

SEQ ID NO: 35 - DNA, 819 bp, Pasteurella multocida
  source 1..819: mol_type = other DNA
atggataaatttgccgaacatgaaattccgaaagccgttattgttgcaggtaatggtgaaagcctgagccagattgattatcgtctgctgccgaaaaactatgatgtgtttcgttgcaaccagttctatttcgaagaacgctattttctgggcaacaaaatcaaagccgtgttttttacaccgggtgtttttctggaacagtattacaccctgtatcatctgaaacgcaacaacgaatatttcgtggataatgtgattctgagcagctttaatcatccgaccgttgatctggaaaaaagccagaaaattcaggccctgtttatcgatgtgattaacggctatgaaaagtacctgagcaaactgaccgcatttgatgtttatctgcgctataaagaactgtatgaaaaccagcgtattaccagcggtgtttatatgtgtgcagttgcaattgccatgggctataccgatatttatctgaccggtatcgatttttatcaggccagcgaagaaaactatgccttcgataacaaaaagccgaacattattcgcctgctgcctgattttcgtaaagaaaaaaccctgtttagctaccacagcaaagatatcgatctggaagcactgagctttctgcagcagcattatcatgttaacttctatagcattagcccgatgagtccgctgagtaaacattttccgattccgaccgtggaagatgattgtgaaaccacctttgttgcaccgctgaaagagaactatattaacgatattctgctgcctccgcacttcgtttatgaaaaactgggcaccattgtgagctaa

SEQ ID NO: 36 - DNA, 903 bp, Campylobacter jejuni
  source 1..903: mol_type = other DNA
atgacacgtacccgtatggaaaatgaactgattgtgagcaaaaacatgcagaacattatcattgcaggtaatggtccgagcctgaaaaacattaactataaacgtctgcctcgcgagtatgatgtttttcgttgtaaccagttctacttcgaggacaaatattacctgggcaaaaaaatcaaagccgtgtttttcaatccgggtgtttttctgcagcagtatcataccgcaaaacagctgattctgaaaaacgagtacgagatcaaaaacatcttttgcagcacctttaacctgccgtttattgaaagcaacgatttcctgcaccagttttacaacttttttccggatgcaaaactgggctatgaagtgattgaaaacctgaaagaattctacgcctacatcaagtacaacgagatctatttcaacaaacgcattaccagcggtgtttatatgtgtgcaattgccattgcgctgggctataaaaccatttatctgtgcggtatcgatttctatgaaggcgacgttatttatccgtttgaagcaatgagcaccaacattaaaacaatctttccgggtatcaaagacttcaaaccgagcaattgtcacagcaaagaatatgatattgaggccctgaaactgctgaaaagcatctataaagtgaacatctatgccctgtgtgatgatagcattctggcaaatcattttccgctgagcattaacatcaacaacaactttaccctggaaaacaagcacaacaacagcattaatgatatcctgctgaccgataatacaccgggtgttagcttttacaaaaatcagctgaaagccgataataagatcatgctgaacttctacaacatcctgcatagcaaagacaacctgatcaaattcctgaacaaataa

SEQ ID NO: 37 - DNA, 780 bp, Campylobacter jejuni
  source 1..780: mol_type = other DNA
atgaagaaagttattattgcaggtaatggtccgagcctgaaagaaattgattatagccgtctgccgaacgattttgatgtttttcgttgcaaccagttctacttcgaggataaatactacctgggcaaaaaatgcaaagccgtgttttataacccgatcctgtttttcgaacagtactataccctgaaacatctgatccagaatcaagagtatgaaaccgaactgattatgtgcagcaattataaccaggcgcatctggaaaatgagaactttgtgaaaaccttctatgactattttccggatgcacatctgggctacgattttttcaaacagctgaaagatttcaacgcctacttcaaatttcacgagatctatttcaatcagcgcattaccagcggtgtttatatgtgtgcagttgcaattgccctgggctataaagaaatttatctgagcggcatcgatttctatcagaatggtagcagctatgcctttgacaccaaacagaaaaatctgctgaaactggcaccgaacttcaaaaatgataacagccattatatcggccacagcaaaaacaccgatattaaagcactggaatttctggaaaaaacctataaaatcaaactgtattgtctgtgcccgaatagcctgctggcaaactttattgaattagcaccgaatctgaacagcaacttcatcatccaagagaagaacaactacaccaaggatattctgattccgagcagcgaagcatatggcaaatttagcaaaaatatcaattaa

SEQ ID NO: 38 - DNA, 2818 bp, Campylobacter jejuni
  source 1..2818: mol_type = other DNA
atgaaagaaataaaaatacaaaatataatcataagtgaagaaaaagcacccttagtcgtacctgaaataggcattaatcataatggcagtttagaactagctaaaattatggtagatgcagcctttagcgcaggtgctaagattataaagcatcaaactcatattgttgaagatgagatgagtaaggccgctaaaaaagtaattcctggtaatgcaaaaataagcatttatgagattatgcaaaaatgtgctttggattataaagatgagctagcacttaaagaatacacagaaaaattaggtcttgtttatcttagcacacctttttctcgtgcaggtgcgaaccgcttagaagatatgggagttagtgcttttaagattggttcaggtgagtgtaataattatccgcttattaaacacatagcagcctttaaaaagcctatgatagttagcacaggaatgaatagtattgaaagtataaaaccaactgtaaaaatcttattagacaatgaaattccttttgttttaatgcacacgaccaatctttacccaaccccgcataatcttgtaagattaaacgctatgcttgagttaaaaaaagaattttcttgtatggtaggcttaagcgaccacacaacagataatcttgcgtgtttaggtgcagttgtacttggagcttgtgtgcttgaaagacattttactgatagtatgcatagaagtggccctgatatagtttgttctatggatacaaaggctttaaaagagctaattatacaaagtgagcaaatggctataataagaggaaataatgaaagtaaaaaagcggctaaacaagaacaagttacaattgattttgcctttgcaagtgtagttagcattaaagatattaaaaaaggcgaagttttatctatggataatatttgggttaaaagacctggacttggtggaattagtgcggctgaatttgaaaatattttaggcaaaaaagcattaagagatatagaaaatgatgctcagttaagctatgaggattttgcgtgaaaaaaatcctttttataacaggctctagggctgattattctaagattaaatctttaatgtacagggtgcaaaactcaagcgaatttgaactttacatctttgcaacaggaatgcacttaagtaaaaattttggctatacagttaaagaactttataaaaatggctttaaaaatatttatgaatttataaattatgataaatattatcaaactgataaggctttagctactacaattgatggattttcaaggtatgcaaatgagctaaaacctgatttaatcgtagtacatggagatagaattgagcctttagcagcagctattgttggagcattaaataatatcttagtagcgcatattgaaggcggagagatttcaggaactattgacgatagcttacgccacgctatatcaaaactagctcatattcatttagtaaatgatgagtttgcaaaaaggcgtttaatgcagcttggagaagatgaaaaatctatttttatcataggttcgcctgatttagaacttttaaacgataataaaatttcacttagcgaagcaaaaaaatattatgatataaattatgaaaactacgctttgcttatgtttcatcctgttacaactgaaattactagcattaaaaatcaagcagacaatttagtaaaagcactgatacaaagtaataaaaattatattgttatttatccaaataatgatttaggttttgaattaatcttgcaaagctatgaagagtttaaaaataaccctagatttaagctttttccatcgcttagatttgagtattttataactttgttaaaaaatgctgattttataataggtaattcaagttgtattttaaaagaggccttatacttaaaaacagcagggattttagttggctcaagacaaaatggaagacttggcaatgaaaatacactaaaagttaatgcaaatagtgatgaaatactaaaagctattaacactattcataaaaaacaagatttatttagcgctaagttagagattttagatagctcaaaattattttttgaatatttacaaagcggagatttttttaaactcagcacacaaaaagtttttaaggatataaaatgagcttagcaataatccctgctcgtggtggctcaaagggtattaaaaataaaaatttggttttattaaacaataaacctttaatttactacacgatcaaagctgcactaaatgctaaaagcattagtaaagttgttgtaagcagtgatagtgatgaaattttaaattatgcaaaaagtcaaaatgttgatattttaaaacgcccaattagccttgcacaagatgataccacaagcgataaagtgctgttacatgctctaaaattttataaagattatgaagatgtagtttttttacaacccacttcaccgctaagaacaaatattcatattaatgaagcttttaatctttataaaaatagcaatgcaaatgccctaattagcgtaagcgaatgtgataataaaattctaaaagcctttgtttgtaatgattgtggcgatttagcagggatttgtaatgatgaatatccttttatgccaaggcaaaaattgcctaaaacttatatgagcaatggtgcaatttatattttaaagataaaagaatttttaaacaatcctagctttttacaaagcaaaaccaagcattttttaatggacgaaagctcaagtttagatattgactgtttggaggatttaaaaaaggttgaacagatatggaaaaaataa

SEQ ID NO: 39 - protein, 332 aa, Neisseria meningitidis
MQPLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRPEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGAWLDFAADGRMRRLFTLRQYFGILHRLLKNR

SEQ ID NO: 40 - protein, 439 aa, Helicobacter pylori
MISVYIISLKESQRRLDTEKLVLESNEKFKGRCVFQIFDAISPKHEDFEKFVQELYDSSSLLKSDWFHSDYCYQELLPQEFGCYLSHYLLWKECVKLNQPVVILEDDVALESNFMQALEDCLKSPFDFVRLYGHYWGGHKTNLCALPVYTETEEAEASIEKTPIENYEVTSPPPPNPTRDTQQDFITETQQDPKELSEPCKIAPQKISFNQVVFKKIKRKLNRFIGSILARTEVYKNIVAKYDDLTTKYDDLTTKYDDLTTKYDDLTTKYDDLNKNIAEKYDELMGKYESLLAKEVNIKETFWESRADSEKEALFLDHFYLTSVYVATTAGYYLTPKGAKTFIEATERFKIIEPVDMFINNPTYHDIANFTYVPCPVSLNKHAFNSTIQNAKKPDISLKPPKKSYFDNLFYHKFNARKCLKAFNKYSKQYAPLKTPKEV

SEQ ID NO: 41 - DNA, 203 bp, synthetic construct
  misc_feature 1..203: note = Promoter Sequence
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaacaaaaaccggagatacc

SEQ ID NO: 42 - DNA, 203 bp, synthetic construct
  misc_feature 1..203: note = Promoter Sequence
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaactgagaaacagct

SEQ ID NO: 43 - DNA, 203 bp, synthetic construct
  misc_feature 1..203: note = Promoter Sequence
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaaggaaaaacagct

SEQ ID NO: 44 - DNA, 203 bp, synthetic construct
  misc_feature 1..203: note = Promoter Sequence
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaccgagaaacagct

SEQ ID NO: 45 - DNA, 310 bp, synthetic construct
  misc_feature 1..310: note = Promoter Sequence
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaactaggaaacagct

SEQ ID NO: 46 - DNA, 203 bp, synthetic construct
  misc_feature 1..203: note = Promoter Sequence
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaagagcaaaacagct

SEQ ID NO: 47 - DNA, 189 bp, synthetic construct
  misc_feature 1..189: note = Promoter Sequence
  source 1..189: mol_type = other DNA
gaaaacattcataaattaaatgtgaattgccgcacacattattaaataagatttacaaaatgttcaaaatgacgcatgaaatcacgtttcactttcgaattatgagcgaatatgcgcgatgcctacaagcatcgtggaggtccgtgactttcacgcatacaacaaacattaaccaaggaggaaacagct

SEQ ID NO: 48 - DNA, 239 bp, synthetic construct
  misc_feature 1..239: note = Promoter Sequence
  source 1..239: mol_type = other DNA
ccatttagccatagtaaaaacatgaattgtttgatttcgcgcatattcgctcataattcgaaagtgaaacgtgatttcatgcgtcattttgaacattttgtaaatcttatttaataatgtgtgcggcaattcacatttaatttatgaatgttttcttaacatcgcggcatgcctacaagcatcgtggaggtccgtgactttcacgcatacaacaaacattaaccaaggaggaaacagct

SEQ ID NO: 49 - DNA, 310 bp, synthetic construct
  misc_feature 1..310: note = Promoter Sequence
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaactgagaaacagct

SEQ ID NO: 50 - DNA, 310 bp, synthetic construct
  misc_feature 1..310: note = Promoter Sequence
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaccgagaaacagct

SEQ ID NO: 51 - DNA, 310 bp, synthetic construct
  misc_feature 1..310: note = Promoter Sequence
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagaaaaacagct

SEQ ID NO: 52 - DNA, 350 bp, synthetic construct
  misc_feature 1..350: note = Promoter Sequence
  source 1..350: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgttttaacgttgtaacccgtatgtaacagtgaataatcacttttgccgaggtaacagcgtcataacaacaattaaagccgttttctggagcgttaccgggcatggaagaacgaattttaaaaagtgagcttcggcgttcagtaacacttcattaactctactgccccgccgagcatttatctcaagcactaccctgcataagcaaggaggaaacagct

SEQ ID NO: 53 - DNA, 310 bp, synthetic construct
  misc_feature 1..310: note = Promoter Sequence
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaaggaaaaacagct

SEQ ID NO: 54 - DNA, 310 bp, synthetic construct
  misc_feature 1..310: note = Promoter Sequence
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagctaaacagct

SEQ ID NO: 55 - DNA, 189 bp, synthetic construct; identical to SEQ ID NO: 47
  misc_feature 1..189: note = Promoter Sequence
  source 1..189: mol_type = other DNA

SEQ ID NO: 56 - protein, 417 aa, Escherichia coli [sequence omitted]